(19) United States
(12) Patent Application Publication
(10) Pub. No.: US 2018/0357886 A1
(43) Pub. Date: Dec. 13, 2018

(54) SYSTEM, DEVICES AND METHODS FOR HEALTH CARE WORKER TRAINING, MONITORING AND PROVIDING REAL TIME CORRECTIVE GUIDANCE FOR PROCEDURES AND PRACTICE RELATED TO HOSPITAL INFECTION CONTROL

(71) Applicants: Isaac TAVORI, Herzelia (IL); Lior FLEISCHER, Tel Aviv (IL)

(72) Inventors: Isaac TAVORI, Herzelia (IL); Lior FLEISCHER, Tel Aviv (IL)

(21) Appl. No.: 15/780,390

(22) PCT Filed: Dec. 1, 2016
(86) PCT No.: PCT/IL2016/051292

§ 371 Date: May 31, 2018

Related U.S. Application Data
(60) Provisional application No. 62/261,322, filed on Dec. 1, 2015.

(51) Int. Cl. G08B 21/24; G06K 9/00; G06K 7/10; G06F 1/16; G16H 40/20

(52) U.S. Cl. CPC G08B 21/245; G06K 9/00355; G16H 40/20; G06F 1/163; G06K 7/10396

(57) ABSTRACT

A system, devices and methods for training of a health care worker, real time monitoring of a health care worker and providing real time corrective guidance for procedures related to hospital infection control. Furthermore, the devices, systems and methods of the present invention are configured to provide monitoring the performance and the quality of a hand hygiene procedure and providing real time, in accordance with the World Health Organization's “5 Moments Hand Hygiene” events protocol, corrective guidance to the health care worker procedures and practice related to hospital infection control.

### FIELD OF THE INVENTION

The present invention relates to devices, systems and methods for infection control practices in health care facilities and more particularly, to devices, systems and methods for training of a health care worker, real time monitoring and providing real time corrective guidance for procedures related to hospital infection control. Furthermore, the devices, systems and methods of the present invention are configured to provide monitoring the performance and the quality of a hand hygiene procedure and providing real time corrective guidance to the health care worker procedures and practice related to hospital infection control.

### BACKGROUND OF THE INVENTION

Infection control practices and actions during patient treatment are essential for protecting patients and Health Care Workers (HCW) from transmission of infection causing microorganisms from one site to another on same patient, from patient to patient, from patient to HCW and from HCW to patient, during patient treatment and stay in health care facilities such as hospitals, clinics, etc. These infection causing microorganisms are transmitted by several routs of transmission that include: reusable medical instruments, medical equipment and surfaces, via hands contacts, through the air, through contaminated and pathogen loaded body fluids, secretions and excretions etc.

In order to prevent the above mentioned transmission of microorganisms, different practices and infection control measures are used by HCW in health care facilities, including disinfection and sterilization of reusable medical devices, isolation protocols for the different routes of transmission, hand hygiene practices, universal precautions, and medical equipment and surfaces cleaning and disinfection. According to common regulations and recommendations, all these infection control practices require training, assimilation, monitoring and periodical training and auditing of the practices in order to maintain a high level of standard of care and promote HCW compliance with these practices and actions.

Hand Hygiene is one of the most important means for reducing the risk of infection transmission in health care facilities. It has been established by years of medical and infection control researches that the level of compliance with hand hygiene is directly connected to the nosocomial infection rate of patients treated within the healthcare system. The World Health Organization (WHO) has published a document on this issue in “WHO_IER_PSP 2009.07_eng.pdf”, and has stressed on the importance of the proper implementation of a proper hand hygiene.

The WHO recommends, as shown in FIG. 1, that any caregiver should perform a hand hygiene process according to the condition of their hands—visibly clean or dirty, before and after the following situations that are referred to by the WHO as the “5 Moments for hand hygiene” chart 20:
1) Before touching a patient (22).
2) Before performing a clean/aseptic procedure (24).
3) After handling body fluids (26).
4) After touching a patient (28).
5) After touching patient surroundings (30).

These required hand hygiene procedures are not standalone procedures in infection control, but rather an essential part of a wider chain of actions dictated by the risks of a specific activity performed on certain patients, for example: if the patient is to be treated by having an IV (intravenous) catheter insertion and the HCW hands are expected to be contaminated with blood, then the HCW is required to perform a hand hygiene to protect the patient from harmful microorganisms and then glove donning to protect himself from contact with the patient's blood. Should this patient had a respiratory transmitted disease, such as TB (tuberculosis), and was put in isolation, the HCW has to use additional protective measures prior to entering the room and approaching the patient. That includes wearing of additional personal protective equipment (PPE) such as a protective gown, a facial mask and/or respirator and protective goggles that had to be worn and removed in a specific defined manner and perform hand hygiene to prevent contamination of the HCW skin or clothing in the process. If during the mentioned procedure some blood was sprayed on the floor or on the patient's bed, a cleaning and disinfection process is required and a cleaning HCW is called, that HCW has to perform the cleaning and disinfection process after performing hand hygiene and protecting himself, in a certain way that ensures that the removal of the contaminant and the proper exposure way and time to the disinfectant.

The required hand hygiene procedures can be divided into two main processes as defined below:
1) A routine hand wash, as shown in FIG. 2a (chart 40), that includes the use of soap and water or an antiseptic detergent solution for performing a hand wash when hands are visibly soiled.
2) A routine hand disinfection with an alcoholic solution before and after every patient contacts as previously detailed, a routine called WHO the “Handrub”, as shown in FIG. 2b (chart 50).

The above mentioned procedures have a descriptive set of actions that defines a good and correct hand hygiene practice according to the purpose as described above.

Two related WHO charts are:
1) Technique for donning and removing non-sterile examination gloves”, as shown in FIG. 3a (chart 60); and
2) Putting on and removing PPE”, as shown in FIG. 3b (chart 70).

The identification of the correct Moment out of “The 5 Moments” and the risks and correct protective measurements that the HCW needs to implement, are the most important steps in infection prevention activities as they define and constitute the need for performing and tacking certain infection control steps and means.

Prior art products that are able to monitor hand hygiene, provide the infection control teams information on the compliance and the frequency of the medical staff performing the hand hygiene procedures prior and after contacting a patient, when Moments 1 and/or 4 of the WHO Moments occur.

Existing methods can provide quantitative data and very limited qualitative data as these methods measure the number and the duration of the process of the hand hygiene procedures performed by the medical staff. These systems cannot provide any indication or information about the quality of the way the hand hygiene procedures were performed or if they were performed at the right moments during the daily tasks. Some existing systems include a detector of a personal identification device that is installed at the room entrance, and on or in a hand hygiene device or place. Some existing systems may contain a patient proximity sensor.

Hence, existing hand hygiene monitoring systems are able to detect the entrance of a care giver (CG) to a patient's room, then detect the performance of a hand hygiene prior to the patient contact or prior to leaving the room or moving to another patient or the lack of them. The existing systems are measuring the duration of the hand hygiene process as well as a limited indication of the quality of the process.

Some existing hand hygiene monitoring systems, for example, the Harmony system described in the publication “Harmony: A Hand Wash Monitoring and Reminder System using Smart Watches” by Mondol et al., utilize a hand bracelet, a smart watch or other type of smart-wrist-wearable device. The Harmony system discloses a hand hygiene performance monitoring system (“The Harmony System”) that provides hand hygiene performance monitoring through hand motions and gestures recognition in accordance to the WHO described procedures. The smart-wrist-wearable device includes an accelerometer and a gyroscope that facilitate identifying and monitoring of the hand hygiene motions described in the WHO “Hand Wash” and/or “Hand RUB” charts. However, these existing hand hygiene monitoring systems concentrate mostly on identifying those specific hand motions for short sampling events, and refer to their presence in the collected signals as a validation of the fact that a hand hygiene is taking place, with no continuous monitoring of the hand motions specifically.

U.S. Pat. No. 8,698,637, by Y. Raichman, disclosed systems, apparatuses and methods for analyzing hygiene habits of a user. The method comprises attaching a personal hygiene monitor to the user, using the attached personal hygiene monitor for detecting a plurality of personal hygiene events related to the user, logging the plurality of personal hygiene events to allow configuring a user hygiene profile of the user, and estimating a hygiene level of one or more hygiene habits of the user according to the user hygiene profile. Raichman does not require both hands to be in close proximity for proper handwash or handrub, as required by the WHO. He further does not differentiate all movements required to perform proper handwash or handrub as required by the WHO, and is not able to differentiate the 5 moments as required by the WHO.

Most hand hygiene monitoring systems only identify the performance of hand hygiene motions, when the HCW approaches the patient and when he/she leaves the patient bed. But, in order to determine if one of the WHO “5 Moments” has occurred, while a HCW is attending a patient, monitoring of hand motions that are not defined as hand hygiene hand motions (herein after referred to as “non-Hand-Hygiene hand motions”) of the HCW is required.

According to a research, “The HOW2 Benchmark Study”, AJIC, February 2011, by Steed et al., the different Moments of the “5 Moments” have different occurrence rate during the daily work. Moments 1 and 4—approaching and leaving the patient's bed account for 49% of the hand hygiene opportunities, while the rest of the Moments account for 51% of the Moments. The opportunities breakdown is as follows: Moment 1=21%, Moment 2=6%, Moment 3=10%, Moment 4=28% and Moment 5=35%. Moments 3 and 5 are the most important Moments from point of view of infection control, as these Moments are the Moments that the HCW are re-contaminated from microorganisms from touching an infected wound or site on the patient's body, or touching the surroundings of a patient. Thereby, such touching increase the risk of transferring those microorganisms to other susceptible sites on the patient. Moments 3 and 5 represent 45% of the opportunities for hand hygiene. The same applies to Moment 2, which is important as well, as this Moment represents the requirement that the hands are safe as aseptic procedures are procedures performed on infection susceptible sites or invasive activities that present opportunities to transmit infections such as IV site infections, UTI, central line infections etc. Moment 2 accounts for 6% of the hand hygiene opportunities.

By monitoring the non-Hand-Hygiene hand motions that existing hand hygiene monitoring systems ignore, the monitoring ability the system can improve substantially and contribute to the infection control and identifying additional hand hygiene opportunities that that prior art systems cannot. Hand hygiene monitoring systems that concentrate only on identifying the performance of the hand hygiene motions at Moments 1 and 4 have a limited contribution to the overall infection control as they ignore the 51% of opportunities for hand hygiene that are required to ensure the patient's safety. The combination of sets of motions such as glove donning, can indicate of Moments 2 and 3 and so on. With the combination of info from different additional sensors a much accurate distinction can be made.

Charts 20, 40 and 60 describe in a precise manner the 5 Moments when an HCW should perform the Hand Hygiene and the way he/she should perform the hand hygiene with an antiseptic or with and alcoholic hand rub (AHRB). As at the 5 Moments, in which the hand hygiene is required, are not always easy to remember by the HCW and the way these HH processes must be done, is not something the HCW can perform without prior proper training.

The HCW needs to be trained to properly identify the 5 different Moments (20) for performing the HH and this knowledge needs to be audited and validate to ensure the performance of the HH when a WHO Moment occur and reduce the risks of nosocomial infections. The same applies to the proper way of performing the HH itself, being it the antiseptic hand wash or the alcoholic hand rub procedure.

The WHO document recommends that a HCW needs to be trained with all the above and be continuously audited and monitored by human observers by using standard forms the WHO included in its document. Currently the training of a HCW is usually done at the arrival to a health institute to start working within a health institute and at situations when after an outbreak is identified in a health institute.

The means that are being currently used or training includes frontal presentations, posters, training interactive software for identifying the 5 Moments and videos. For hand hygiene performance training and auditing they use a black light box together with an AHRB that contains a UV indicative substance. After the training HCW performs hand hygiene with the tinted AHRB they place their hands into the box with UV light and check if they have covered all hand surfaces properly according to the hand coverage with the fluorescence for performance validation. Today there is on the market a system named Hand-in-Scan that is an automated hand fluorescence scanner after the HCW used a tinted AHRB.

One of the most important tasks of any infection control team in a health care facility is to promote and improve hand hygiene compliance. The WHO, the CDC and other professional bodies have stated that the improvement of hand hygiene compliance can lead to up to 72% reduction in hospital acquired infections HAI and save the lives of over 1.5 million people worldwide that die every year of HAI. It can reduce morbidity too in several million people that acquire nosocomial infections while being treated in those healthcare facilities for other conditions. Currently the average hand hygiene compliance in many health care facilities around the world is under 40% and the WHO the CDC and other professional bodies world-wide are seeking for methods to improve and increase the hand hygiene compliance rate all around the world. The currently used methods to improve hand hygiene compliance includes HCW training by infection control teams, HCW monitoring by infection control teams or through electronic monitoring technologies.

There is therefore a need and it would be advantageous to have an infection-control-practices-monitoring system that utilizes a smart-wrist-wearable device, and that can identify the performance of the hand hygiene motions at all 5 Moments (20). There is a further need to provide a comprehensive real time HCW training, monitoring and corrective guidance for the following infection control practices, hand hygiene (20, 40, 50), glove donning/removal (60), isolation and Personal Protective Equipment (PPE) wearing/removal (70), environmental cleaning and disinfection, based on the WHO and other infection control policy establishing bodies recommended practices and process.

There is also a need for means and methods for enhancing the scope and quality of infection control practices and actions during patient treatment, as well as raising and maintaining the awareness for these practices by the stuff of care givers.

### SUMMARY OF THE INVENTION

As infection control practices and processes are performed by the hands of a HCW's, there is provided herein a device, system and method that are based on hand motions detection and interpretations that enables the training, monitoring, providing real-time corrective guidance, auditing and validating the way these infection control practices and processes are performed. It is an intention of the present invention to improve compliance, quality of care and reduce the risks of acquiring infections by the patients or HCW.

The present invention is based on the fact that each and every usage of the recommended isolation items such as face masks gowns, goggles, gloves, hand hygiene, is performed by the hands with a specific sequence and hand motions that are important for infection control and prevention of accidental contamination, as can be seen from the attached WHO recommended protocols.

A principle intention of the present invention includes providing a comprehensive infection-control-practices-monitoring system that utilizes a smart-wrist-wearable device, and that can identify the performance of the hand hygiene motions at all 5 Moments (20). The infection-control-practices-monitoring system further provides a comprehensive real time HCW training, monitoring and corrective guidance for the following infection control practices, hand hygiene, glove donning/removal, isolation and Personal Protective Equipment (PPE) wearing/removal, environmental cleaning and disinfection, based on the WHO and other infection control policy establishing bodies recommended practices and process.

Contrary to prior art technologies, by monitoring the additional hand motions that are non-Hand-Hygiene related motions, the ability of the infection-control-practices-monitoring system to differentiate between the WHO “5 Moments for Hand Hygiene” (20) is expanded, and overlapping Moments that do not require additional hand hygiene, when moving from one patient to another, can be avoided. In order to identify non-Hand-Hygiene hand motions that can indicate with good accuracy if additional hand hygiene is required on top of the hand hygiene performed by the HCW, when approaching a patient bed (Moment 1, 22) a detected non-Hand-Hygiene hand motion is matched against a database of non-Hand-Hygiene hand motions.

Typically, the database of non-Hand-Hygiene hand motions may include, but not limited to, different standard activities that a HCW may perform when attending a patient's bed side and which activities require the performance of additional hand hygiene procedures. The identification of non-Hand-Hygiene hand motions may be validated using additional sensors such as a proximity sensor placed in the wrist band, an IR/Video sensor in the Personal-Communication-Monitoring-Badge (PCMB) combined with the detected area of the patient's body where at the HCW hands were detected, shifting privacy curtains, glove donning and so on, as described in the following non limiting examples for each of the WHO Moments:

Moments 2 (24) and 3 (26) examples: The HCW is approaching the patient's bed and performs a hand hygiene then a glove donning action (being a non-hand hygiene hand set of motions), wherein hand motions of that action are detected. The infection-control-practices-monitoring system assumes that the HCW intends to perform an aseptic procedure (Moment 2 (24)) on the patient or a procedure that may expose himself/herself to body fluids or secretions (Moment 3 (26)). Although from the point of view of hand hygiene in both cases hand disinfection is indicated, after removing the gloves, the two actions can be differentiated by data obtained from other sensors such as the HCW hand positioning from the IR/Video sensor on the PCMB, a proximity sensor to body parts from the smart-wrist-wearable device. If the PCMB indicates that the HCW hands are performing a procedure on the upper part of the patient's body and the hand is in proximity to the patient skin, and moving with short and delicate motions, then Moment 2 (24) can be assumed and reported to the infection control. If Moment 2 (24) is assumed through analyzing the hand motions of glove donning and proper hand hygiene was not detected prior to glove donning or after the glove removal, then a failure to comply is reported and stored on the HCW file for further analysis by the infection control team. If the signal from the PCMB IR/Video sensor indicates that the positioning of the HCW is on the lower part of the patient's body at a distance from the patient silhouette at the bed side and no patient's skin is detected by the proximity sensor, then the infection-control-practices-monitoring system assumes Moment 3 (26) and anticipates a hand hygiene at the end of the procedure.

Moment 5 (30) examples: Moment 5 (30) is about touching things in the surroundings of the patient. With regards to Moment 5 (30), the database of non-Hand-Hygiene hand motions may include the following hand and arm motions: puling a privacy curtain, leaning against bed side rails, leaning on a patient's bed, pulling an IV pole, pulling or touching a piece of medical equipment (ECG, respirator, etc. communicated, for example, via IOT)), touching an assistance button, touching a patient chart, typing data into a computer, touching a patient's pillow or blanket, turning ON/OFF the light, touching a bed controller, etc.

When the HCW is approaching the patient's bed he/she is required to perform a proper hand hygiene. While performing the hand hygiene, hand motions are detected by the infection control practices monitoring system (ICPMS) to be at the side of the body. The HCW needs to perform a body examination and needs to close the privacy curtain. At this moment there are 2 possibilities: if the HCW uses the hand wearing the smart-wrist-wearable device, the accelerometer and the gyroscope detect the changes in the hand positioning and being higher than the other hand that is in a resting position, and then detecting a long puling motion at the same level for a while. The second possibility is that the HCW is using the hand without the smart-wrist-wearable device. In this case, the smart-wrist-wearable device detects a pivotal motion at approximately the same height when the hand was in the resting position and then a linear motion surrounding the bed. Additional data from the PCMB IR/Video sensor is used to validate these motions. This HCW contact with the privacy curtain is assumed by the infection-control-practices-monitoring system to be a contact with the patient's surrounding, meaning Moment 5 (30). Therefore, a second hand hygiene is indicated prior to the HCW contacting the patient, according to the WHO “5 Moments” (20). If an additional proper hand hygiene procedure is detected, then the infection-control-practices-monitoring system reports, for example to the infection control DB on hospital servers, that a hand hygiene process was performed in accordance with Moment 5 (30) requirement. If an additional proper hand hygiene procedure is not detected, then the infection-control-practices-monitoring system reports to the infection control that a hand hygiene process was not performed in accordance with Moment 5 (30) requirement. If it was wrongly performed, then the infection-control-practices-monitoring system provides an indication to the HCW and reports to the HCW file for further analysis by the infection control team.

The above examples demonstrate the differences between the infection-control-practices-monitoring system and prior art systems, which prior art systems monitor only the hand hygiene motions performed at 2 opportunities (Moments 1 (22) and 4 (28)) out of the 5 required by the WHO, while the infection-control-practices-monitoring system of the present invention is configured to monitor hand motions at all 5 WHO moments through monitoring the hand hygiene hand motions through all the procedure and the non-Hand-Hygiene motions in combination with additional data from additional sensors and/or detected actions.

According to the teachings of the present invention, there is provided an infection-control-practices-monitoring system for monitoring hand motions of a health care worker (HCW), the hand motions are related to infection control practices. The system includes at least one smart-wrist-wearable device adapted to be worn on a human wrist of a first hand; a bracelet-communication configured to communicate with any device sharing same the communication protocols; and a data repository unit.

The at least one smart-wrist-wearable device includes a living body proximity sensor, an accelerometer, a gyroscope and a communication unit for communicating with the data repository unit.

The living body proximity sensor is configured to detect a live tissue at a short range of up to 50 centimeters.

The at least one of the accelerometer, the gyroscope and the living body proximity sensor detects hand motions of the health care worker.

The smart-wrist-wearable device is configured to perform a hand motion analysis to thereby analyze the detected hand motions of the health care worker, and to thereby determine if the hand motion were performed in compliance with a preset required hand motion sequence and rate.

The data repository unit may include a database of known hand motions, and wherein the hand motion analysis includes matching the detect hand motions with the known hand motions.

Optionally, the at least one smart-wrist-wearable device further includes a skin and muscle motion detector for detecting under-skin muscle motion.

Optionally, the at least one smart-wrist-wearable device further includes a RFID reader.

Optionally, the at least one smart-wrist-wearable device further includes an indicator.

Optionally, the skin and muscle motion detector include segmented inflatable inner lining elements, wherein the at least one smart-wrist-wearable device embraces the external surface of the wrist of the HCW with soft tightness, such that the inner lining elements can sense the skin and the under-skin muscle motion.

Optionally, the living body proximity sensor is selected from group including a thermal sensor, an RLC sensor, an ultrasound sensor and a combination thereof.

Optionally, the living body proximity sensor is capable of sensing the second hand of the HCW.

Preferably, the infection-control-practices-monitoring system further includes a personal-communication-monitoring-badge device having a processing unit; a PCMB-communication unit that is configured to communicate with any device sharing same the communication protocols; an indicator; and an RFID reader.

The processing unit is configured to analyze the detected hand motions of the health care worker to thereby determine if the hand motion were performed in compliance with a preset required hand motion sequence and rate.

The PCMB-communication unit is configured to communicate with the at least one smart-wrist-wearable device and the data repository unit.

The data repository unit includes a data base of known hand motions, wherein hand motion analysis includes matching the detect hand motions with the known hand motions.

The personal-communication-monitoring-badge device is configured to perform a hand motion analysis to thereby analyze the detected hand motions of the health care worker, and to thereby determine if the hand motion were performed in compliance with a preset required hand motion sequence and rate.

Optionally, the at least one personal-communication-monitoring-badge device further includes an imaging device.

Optionally, the at least one personal-communication-monitoring-badge device further includes a UV emitting light.

Optionally, the at least one personal-communication-monitoring-badge device further includes a UV sensor.

Optionally, the imaging device is an IR imaging device.

Optionally, the infection-control-practices-monitoring system of claim 13, wherein the IR imaging device includes a wide angle lens.

The infection-control-practices-monitoring system may further include at least one Hand Hygiene bottle containing Hand Hygiene solution, wherein the Hand Hygiene bottle includes an RFID tag, and wherein the RFID reader of the smart-wrist-wearable device is configured to read the RFID tag of the Hand Hygiene bottle.

The infection-control-practices-monitoring system may further include at least one Hand Hygiene bottle containing Hand Hygiene solution, wherein the Hand Hygiene bottle includes an RFID tag, and wherein the RFID reader of the personal-communication-monitoring-badge device is configured to read the RFID tag of the Hand Hygiene bottle.

The at least one smart-wrist-wearable device includes a living body proximity sensor, an accelerometer, a gyroscope and a communication unit for communicating with the personal-communication-monitoring-badge device.

The infection-control-practices-monitoring system preset required hand motion sequence and rate are selected from the group of procedures put forward by the World Health Organization” (WHO), including “Your 5 Moments Hand Hygiene”, “How to Handwash”, “How to Handrub”, “technique for donning and removing non-sterile examination gloves” and “Putting on and removing PPE”.

According to further teachings of the present invention, there is provided a smart-wrist-wearable device for detecting and monitoring hand motions of a health care worker (HCW), the hand motions are related to infection control practices. The smart-wrist-wearable device includes an accelerometer; a gyroscope; a living body proximity sensor; and d) a bracelet-communication unit.

The at least one smart-wrist-wearable device adapted to be worn on a human wrist of a first hand;

The bracelet-communication unit is configured to communicate with a data repository unit.

The living body proximity sensor is configured to detect a live tissue at a short range of up to 50 centimeters.

At least one of the accelerometer, the gyroscope and the living body proximity sensor detects hand motions of the health care worker.

The smart-wrist-wearable device is configured to perform a hand motion analysis to thereby analyze the detected hand motions of the health care worker, and to thereby determine if the hand motion were performed in compliance with a preset required hand motion sequence and rate.

Optionally, the data repository unit includes a data base of known hand motions, and wherein the hand motion analysis includes matching the detect hand motions with the known hand motions.

Optionally, the at least one smart-wrist-wearable device further includes a skin and muscle motion detector for detecting under-skin muscle motion.

Optionally, the at least one smart-wrist-wearable device further includes a RFID reader.

Optionally, the at least one smart-wrist-wearable device further includes an indicator.

The smart-wrist-wearable device may further include a UV sensor.

Optionally, the skin and muscle motion detector include segmented inflatable inner lining elements, wherein the at least one smart-wrist-wearable device embraces the external surface of the wrist of the HCW with soft tightness, such that the inner lining elements can sense the skin and the under-skin muscle motion.

Optionally, the living body proximity sensor is selected from group including a thermal sensor, an RLC sensor, an ultrasound sensor and a combination thereof.

Optionally, the living body proximity sensor is capable of sensing the second hand of the HCW.

Optionally, the preset required hand motion sequence and rate are selected from the group of procedures put forward by the World Health Organization” (WHO), including “Your 5 Moments Hand Hygiene”, “How to Handwash”, “How to Handrub”, “technique for donning and removing non-sterile examination gloves” and “Putting on and removing PPE”.

According to further teachings of the present invention, there is provided a personal-communication-monitoring-badge device for monitoring hand motions of a health care worker (HCW), the hand motions are related to infection control practices. The personal-communication-monitoring-badge device includes a processing unit; a PCMB-communication unit; an indicator; and an RFID reader.

The processing unit is configured to analyze the detected hand motions of the health care worker to thereby determine if the hand motion were performed in compliance with a preset required hand motion sequence and rate.

The PCMB-communication unit is configured to communicate with at least one smart-wrist-wearable device and a data repository unit.

The at least one smart-wrist-wearable device adapted to be worn on a human wrist of a first hand and is configured to communicate with the PCMB-communication unit.

The bracelet-communication unit includes a multiplicity of sensors configured at least detect hand motions of the HCW and proximity of the two wrists of the HCW;

The data repository unit includes a data base of known hand motions, wherein hand motion analysis includes matching the detect hand motions with the known hand motions.

The personal-communication-monitoring-badge device is configured to perform a hand motion analysis to thereby analyze the detected hand motions of the health care worker, and to thereby determine if the hand motion were performed in compliance with a preset required hand motion sequence and rate.

Optionally, the at least one personal-communication-monitoring-badge device further includes an imaging device.

Optionally, the at least one personal-communication-monitoring-badge device further includes a UV emitting light.

Optionally, the at least one personal-communication-monitoring-badge device further includes a UV sensor.

Optionally, the imaging device is an IR imaging device.

Optionally, the IR imaging device includes a wide angle lens.

According to further teachings of the present invention, there is provided an infection-control-practices-monitoring method for monitoring hand motions of a HCW while performing a Hand Hygiene (HH) according to the WHO Handwash/Handrub procedures. The method including the steps of:

a) providing an infection-control-practices-monitoring system having a personal-communication-monitoring-badge device;

b) identifying the patient's room;

c) determining that the HCW has used an HH solution;

d) monitoring hand motions of the HCW performing the Hand Hygiene, in each step of the HH procedure;

e) determining compliance with the WHO Handwash/Handrub procedure;

f) providing an indication to the HCW and the patient to indicate success or failure of the HH procedure; and

g) recording to the performed HH procedure at the data repository unit.

The determining compliance motions includes determining proximity of the two wrists of the HCW, while performing the Hand Hygiene procedure.

Optionally, the monitoring hand motions includes all motions required by the WHO Handwash/Handrub procedures, while performing the Hand Hygiene procedure.

According to further teachings of the present invention, there is provided an infection-control-practices-monitoring system having a personal-communication-monitoring-badge device, and further including a training sub-system. The training sub-system includes a computerized device configured to track the training progress and performance level of Hand Hygiene procedures of a multiplicity of health care workers; at least one indicator; a Hand Hygiene bottle having an RFID tag; a Hand-Hygiene hand motions database associated with the data repository unit; a non-Hand-Hygiene hand motions database associated with the data repository unit; and a health-care-workers database associated with the data repository unit.

The computerized device includes a machine learning module that is configured to learn a particular HCW's variations of hand motions.

The machine learning module is configured to track and evaluate the progress of a HCW.

The computerized device is configured to communicate with the PCMB-communication unit and with the data repository unit and the at least one smart-wrist-wearable device.

Optionally, the machine learning module forms a uniquely identifiable hand motion model associated with a particular HCW, based on that HCW specific hand hygiene motions or combination of some of that HCW specific hand hygiene motions.

The infection-control-practices-monitoring system may further include a second smart-wrist-wearable device adapted to be worn on a human wrist of the second hand.

The infection-control-practices-monitoring system may further include an alcoholic hand rub bottle (AHRB) usage sensor for determining that the nozzle of the AHRB is being used.

The infection-control-practices-monitoring system may further include a training module selected from the group of the following modules: frontal presentations, posters, training interactive software for identifying the 5 Moments and videos.

The infection-control-practices-monitoring system may further include a training-motivation game for enhancing HH compliance and motivation to train and improve performance.

Optionally, the game includes a Real Life module, reflecting the training/real life performance level of a HCW, and a Virtual Life module, reflecting the standings of that HCW in the game, wherein the desire to succeed in the game enhances the motivation of that HCW to train and thereby improve the real life performance level of that HCW.

According to further teachings of the present invention, there is provided an infection-control-PPE-monitoring-and-training method for monitoring hand motions of a HCW while putting on and removing personal protective equipment (PPE) according to the WHO PPE procedures.

According to further teachings of the present invention, there is provided an infection-control-glove-monitoring-and-training method for monitoring hand motions of a HCW while donning and removing non-sterile examination gloves according to the WHO glove procedures.

### BRIEF DESCRIPTION OF THE DRAWINGS

The present invention will become fully understood from the detailed description given herein below and the accompanying drawings, which are given by way of illustration and example only, and thus not limiting in any way, wherein:

FIG. 1 (prior art) illustrates the WHO “5 Moments for hand hygiene” chart.

FIG. 2a (prior art) illustrates the WHO “How to Handwash” chart.

FIG. 2b (prior art) illustrates the WHO “How to Handrub” chart.

FIG. 3a (prior art) illustrates the WHO gloves donning and removing chart.

FIG. 3b (prior art) illustrates the WHO PPE Putting-on chart.

FIG. 3c (prior art) illustrates the WHO PPE removing chart.

FIG. 4 is a schematic system diagram illustration of an infection-control-practices-monitoring system, according to embodiments of the present invention.

FIG. 5 is a schematic illustration of a personal-communication-monitoring-badge (PCMB) device for hand hygiene (HH) monitoring and infection control, according to embodiments of the present invention.

FIG. 6 is a schematic illustration of a smart-wrist-wearable device for hand motion monitoring, according to embodiments of the present invention.

FIG. 7 is a schematic flow chart showing an exemplary basic process for detecting hand motions, determining the performance and the quality of a hand hygiene procedure and providing real time corrective guidance to the health care worker performing the hand hygiene procedure, according to embodiments of the present invention.

FIG. 8 is a schematic flow chart showing an exemplary 5-moments-motion-detection process for identifying all of the WHO “5 moments” that require performing the WHO hand hygiene procedures, according to embodiments of the present invention.

FIGS. 9a, 9b and 9c combine to show a schematic flow chart of an exemplary HH-hands-motions-detection process, according to embodiments of the present invention.

FIG. 10 is a schematic flow chart showing an exemplary RFID-tagging process 900 for tagging HH solution bottle and patient, according to embodiments of the present invention.

FIG. 11 is a schematic simplified hand motion model shown in 2D configuration such as a circle or an ellipse positioned in space is described, using Cartesian coordinate system (XYZ) modified when necessary with Euler angles, as a no limiting example, opening of bed-side curtains.

FIG. 12 is a twofold sample diagram of the EEMs of two hospital cleaning fluids: NaDCC and Chlorine Dioxide.

FIGS. 13a and 13b are schematic illustrations of the right and wrong, respectively, of recommended hand motions for contaminated surface cleaning.

FIG. 14 is a schematic flow chart showing an exemplary cleaning-and-disinfecting process for determining that a HCW performed the cleaning-and-disinfecting process properly, according to embodiments of the present invention.

FIG. 15 is a schematic system diagram illustration of a basic infection-control-practices-training system (ICPTS), according to the embodiments of the present invention.

FIG. 16 is a schematic flow chart showing an exemplary cleaning-and-disinfecting process for determining that a HCW performed the cleaning-and-disinfecting process properly, according to embodiments of the present invention.

FIG. 17 illustrates a horse shoe shaped spring that closes an electrical circuit when pressed fully down, to thereby indicate that the bottle's nozzle is being used.

FIG. 18 is a schematic flow chart showing an exemplary an inversed-simulator process for transferring performance and quality achievements of a HCW form “real life” to “virtual life”, according to embodiments of the present invention.

FIG. 19 is a schematic system diagram illustration the motivation relationship between the HCWs and the Infection-control-practices-training/monitoring systems.

FIG. 20 is a schematic system diagram illustration of a basic “The Saving Lives Game”, according to the embodiments of the present invention.

FIG. 21 illustrates iconic group of drawings used in Table 1.

FIG. 22 illustrates iconic group of drawings used in Table 2.

FIG. 23 illustrates iconic group of drawings used in “The Saving Lives Game”.

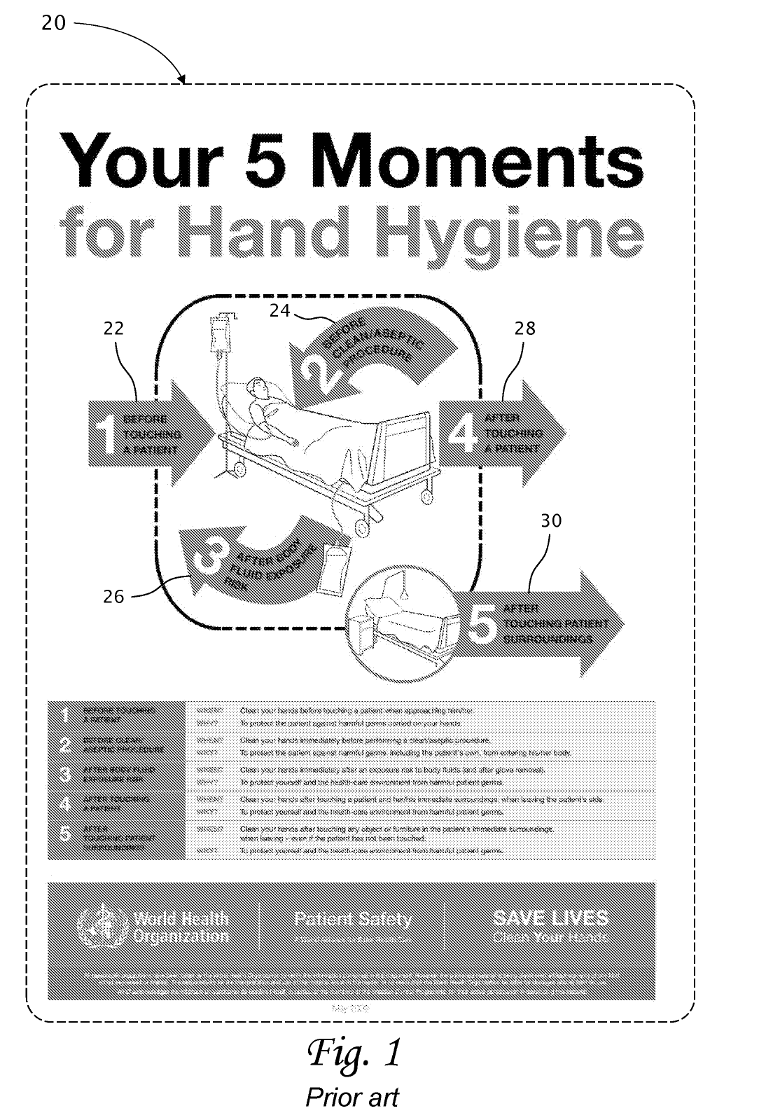

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein; rather, these embodiments are provided, so that this disclosure will be thorough and complete, and will fully convey the scope of the invention to those skilled in the art.

An embodiment is an example or implementation of the invention. The various appearances of “one embodiment,” “an embodiment” or “some embodiments” do not necessarily all refer to the same embodiment. Although various features of the invention may be described in the context of a single embodiment, the features may also be provided separately or in any suitable combination. Conversely, though the invention may be described herein in the context of separate embodiments for clarity, the invention may also be implemented in a single embodiment.

Reference in the specification to “one embodiment”, “an embodiment”, “some embodiments” or “other embodiments” means that a particular feature, structure, or characteristic described in connection with the embodiments is included in at least one embodiment, but not necessarily all embodiments, of the inventions. It is understood that the phraseology and terminology employed herein are not to be construed as limiting and are for descriptive purposes only.

Meanings of technical and scientific terms used herein are to be commonly understood as to which the invention belongs, unless otherwise defined. The present invention can be implemented in the testing or practice with methods and materials equivalent or similar to those described herein.

It should be noted that orientation related descriptions such as “bottom”, “up”, “upper”, “down”, “lower”, “top” and the like, assumes that the associated item is operationally situated.

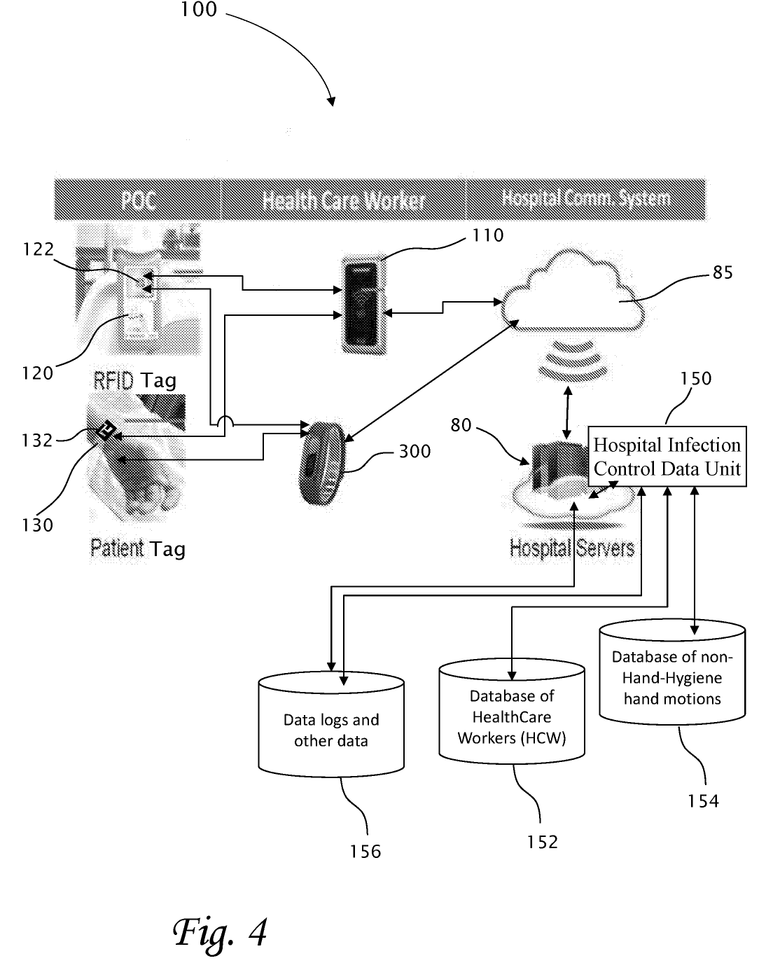

Reference is made to the drawings. FIG. 4 is a schematic system diagram illustration of a basic infection-control-practices-monitoring system 100, according to the embodiments of the present invention. In the example shown in FIG. 4, infection-control-practices-monitoring system 100 includes a computerized device 110 such as, with no limitation, a tablet, a local communication network 85 facilitating computerized device 110 to communicate with an infection control unit 150 of the health facility servers 80, a smart-wrist-wearable device 300, preferably at least one hand hygiene (HH) solution bottle (also referred to as a dispenser) 120 having a RFID tag 122 associated therewith and a RFID tag 132 associated with the patient attached, for example on a patient label 130. Computerized device 110 is associated with a particular HCW and is coupled to operate with that HCW. Infection control unit 150 further includes a health-care-workers database 152, a non-Hand-Hygiene hand motions database 154 and a Hand-Hygiene hand motions database 158. Communication and association refers to establishing communication Protocol, uploading and downloading of data, to and from any of system components. Database 156 includes data logs and other data. Database 156 may further include information related to other hospital equipment, as non-limiting example ECG, medical equipment cleaning and disinfection apparatus, dialysis imaging systems and the like. such an equipment can indicate to HCW required precessions, hence providing Internet Of Things (TOT). Hand-Hygiene hand motions database 158 includes the basic hand motion patterns that combine to a set of basic Hand-Hygiene hand motions that complies with the WHO Handwash/Handrub procedures.

It should be noted that HH solution bottle 120 is coupled with a specific patient, and typically, it is removably attached to the patient's bed. Therefore, identifying the HEI solution bottle 120 means also identifying the patient.

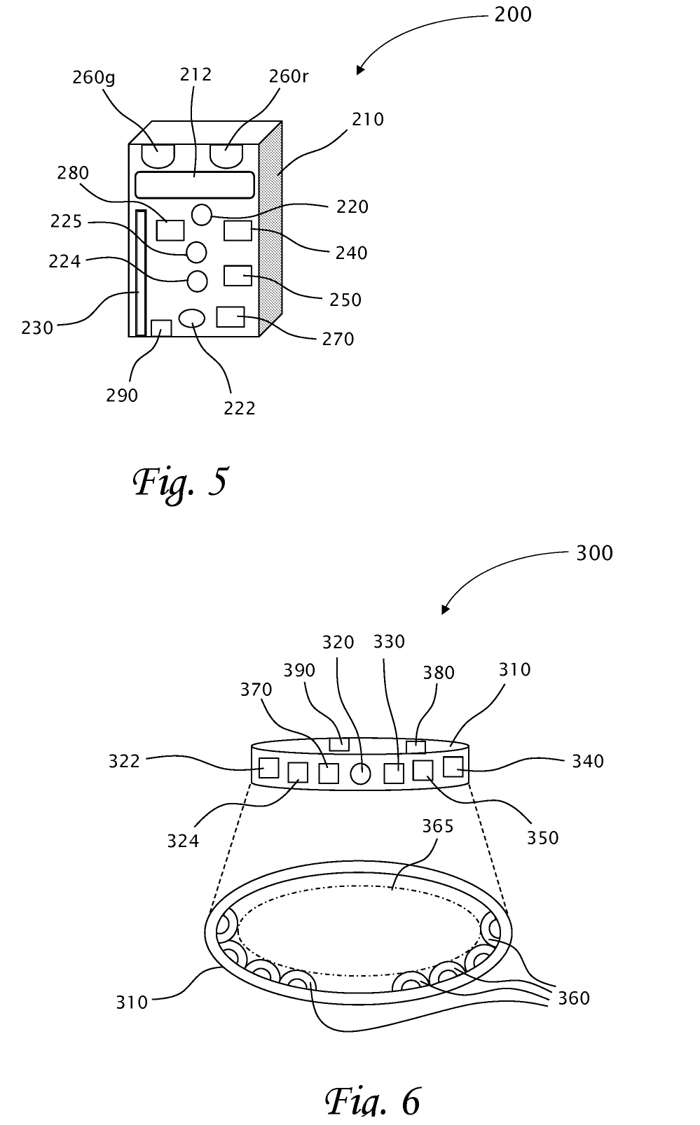

Referring also to FIG. 5, a schematic illustration of a personal-communication-monitoring-badge (PCMB) device 200 for hand hygiene monitoring and infection control, according to embodiments of the present invention, is shown. PCMB device 200 exemplifies a variation of computerized device 110. The present invention will now be described in terms of computerized device 110 being PCMB device 200, but the present invention may user other computerized devices, including tablets, laptops and smart phones. The hand hygiene monitoring is closely related to the WHO “5 Moments for hand hygiene” shown in chart 20, the WHO “How to Handwash” procedure shown in chart 40, and the WHO “How to Handrub” procedure shown in chart 50.

PCMB device 200 includes housing 210, preferably at least one imaging sensor 220, means for reading personal identification data of the HCW coupled to operate with that PCMB device 200, an RFID detector (reader) 240, communication means 250 for communicating with the infection control unit 150 of the health facility servers 80, GO/NO GO indication means such as with no limitations, red and green led lights 260, a data logging module 280 and an operation power, such as rechargeable batteries 290. PCMB device 200 may further include a display 212. RFID reader 240 may be embodied as a physically separate device, and not part of PCMB device 200.

The means for reading personal identification data of the HCW may be an ID card reader slot 230. The communication means 250 are selected from a group including RF, GPRS, BT and Wi-Fi. The imaging sensor 220 are selected from a group sensors including an IR imaging device, an image sensor and an un focused imaging device.

PCMB device 200 may further include other sensors 270, such as an ambient temperature sensor and a GPS.

It should be noted that PCMB device 200 is attached to the carrying HCW at his/her front such that the image sensors 220 are not obstructed.

Typically, non-Hand-Hygiene hand motions database 154 includes different standard activities that a HCW may perform when attending a patient's bed side and which activities require the performance of additional hand hygiene procedures. The identification of non-Hand-Hygiene hand motions may be validated using additional sensors such as a proximity sensor 320 placed in the wrist band, an IR/Video sensor in PCMB 200 combined with the detected area of the patient's body where at the HCW hands were detected, shifting privacy curtains, glove donning and so on. Non-Hand-Hygiene hand motions database 154 may include the following hand and arm motions features: puling a privacy curtain, leaning against bed side rails, leaning on a patient's bed, pulling an IV pole, pulling or touching a piece of medical equipment (ECG, respirator, etc. communicated, for example, via IOT), touching an assistance button, touching a patient chart, typing data into a computer, touching a patient's pillow or blanket, turning ON/OFF the light, touching a bed controller, etc.

Referring also to FIG. 6, a schematic illustration of smart-wrist-wearable device 300, being a hand motion detecting device. Smart-wrist-wearable device 300 includes a wrist attachment means such as, with no limitations, elastic strap 310, preferably a living body proximity sensor 320, an accelerometer 322, a gyroscope 324, an RFID detector (reader) 330, a time measuring unit 340, a communication unit 350 for communicating with PCMB device 200, optionally a skin and muscle motion detector 360 and an operation power, such as rechargeable batteries 390.

Smart-wrist-wearable device 300 may further include other sensors 370, such as a magnetometer. While accelerometer 322 measures body movement and gyroscope 324 measures rotational motion, the magnetometer measures changes in motion directions. Smart-wrist-wearable device 300 may further include an ambient temperature sensor, a skin conductance sensor, an inertial measurement unit (IMU), stretch and distortion sensors, and a skin temperature sensor 380.

The communication means 350 are selected from a group including RF, BT and Wi-Fi. The living body proximity sensor 320 may be, with no limitations, a temperature sensor such as Amphenol ZTP-115. Furthermore, Arduino and Raspberry Pie provide an open source for a complete system components and sensors.

Skin and muscle motion detector 360 may include segmented inflatable inner lining elements for skin and muscle motion detection. In such embodiment, elastic strap 310 embraces the external surface (denoted by broken line 365) of the wrist of the wearing HCW with soft tightness, such that inner lining elements can sense proximal skin and muscle motion, including motion direction, and report to electronics incorporated into elastic strap 310, or to PCMB device 200.

Living body proximity sensor 320 is configured to detect a live tissue at a short range of up to 50 centimeters, and possibly more. Such living body proximity sensor 320 can be used for example while monitoring execution of the WHO Handwash and Handrub procedures. Since these procedures involve the usage of both hands, if the smart-wrist-wearable device 300 is worn only on one hand (referred to herein as the directly monitored hand), prior art monitoring systems simply ignore the other hand (referred to herein as the non-monitored hand) motion. Detecting the continuity in presence of the non-monitored hand, using living body proximity sensor 320, can provide data as to the position of the directly monitored hand relative to the non-monitored hand, and thereby conclude if the procedure is performed as instructed or not. It should be noted that there is no way to perform hand wash or hand rub without having both hands in close proximity, at least during part of the washing process.

Out of the many variations to embody infection-control-practices-monitoring system 100, by a way of example, infection-control-practices-monitoring system 100 operates in the follows: when a HCW enters a patient room within the hospital the GPS/GPRS/Wi-Fi positioning/RFID detector 240 detects the event and turns ON the red indicator light 260r on PCMB device 200 and optionally, provides the HCW a signal, such as an auditory signal or vibratory signal, that reminds him the perform a hand hygiene (HH) prior to approaching a patient. As the HCW approaches the HH bottle 120, smart-wrist-wearable device 300 reads the signal from RFID tag 122 on HH bottle 120 and triggers smart-wrist-wearable device 300 to hand motion detection. The sensors within the smart-wrist-wearable device 300 start detecting the set of hand motions performed by the HCW during the HH. The obtained signals from the sensors are analyzed to derive a certain pattern of motions into a defined behavior or action.

The analyzing module, used to analyze the hand motions is based on the set of motions required by the WHO to be performed during the 2 different HH procedures. When the HH is performed in accordance to the required hand motions and in accordance to the required time duration, then the red led light indicator 260r is turned OFF, the green led light indicator 260g is turned ON and data logging module 280 is updated. If the HH process was not done according to the recommended set of motions, or the time duration was shorter than recommended or the used volume of HH solution was less than required, the process is considered as a failure, the HCW is given an indication signal to perform the HH process again, the indicator red led light 260r stays ON and data logging module 280 is updated. After the HH process is performed correctly and the HCW approaches the patient, the patient's optional RFID is detected and the optional IR imaging device 220 is activated to monitor the activities performed by the HCW on the patient or in his/her surroundings. For example, a wide angle IR camera 220, a passive infrared (PIR) sensor and/or a thermopile are able to include in the field of view the hands coming to a HH bottle 120. When performing HH, either with alcoholic solution or hand wash with water, the temperature of hands changes, and combined with local proximity indicator to the HI-1 solution bottle 120, or local water faucet, it is assumed that the HH is performed. The quality of the HI-1 operation is recorded by the smart-wrist-wearable device 300, and provides indications in accordance with the WHO's recommendations.

The IR camera 220 detects when the HCW moves from one activity to another in a different part of the patient's body. When the HCW moves from one part to another, the green light indicator 260g is turned OFF, the red light indicator 260r is turned ON and the HCW is provided with a signal that reminds him/her to perform a HH. If the HH is performed correctly than the green light indicator is turned ON and the red light turned OFF and data logging module 280 is updated. If the HH is not performed data logger 280 is also updated. The same process happens when the HCW moves to another site or patient and so on.

When the HCW leaves the room even if he/she has performed a HH, data logging module 280 is updated and the red light indicator 260r is turned ON—the default. Indicator lights 260 are visible to the HCW and to the patient and in combination with patient education to watch the lights and observe that the HCW performs a HI-1 prior to approaching him or moving from one action to the other.

The incorporation of the IR video camera 220 or video camera enables infection-control-practices-monitoring system 100 to monitor the actions of the HCW situated adjacent to a patient's bed, and the performance of HI-1 between actions, while performing more than one procedure. The IR camera 220 and the IR temperature sensor that can detect heat generated from live tissue enables infection-control-practices-monitoring system 100 to validate the performance of the HI-1 by detecting the temperature changes of the hand's skin as a result of the exposure to the alcoholic solution or the hand wash with soap and water. Preferably, IR camera 220 and PIR/ultrasound sensors are based on a wide angle lens with an angle that enables the monitoring of the hands at a close distance to a living body or a part thereof.

### Modeling the Human Hand

Modeling the human hand, from shoulder to wrist, yields a seven axes of motion. For each arm motion, Euler joint angles for the seven axes of the human arm can be calculated based on the Cartesian coordinates of each axis. This transformation The seven axes are: shoulder XYZ, ShX, ShY, ShZ, Elbow, ElY, wrist, WrX, WrY, WrZ.

V (Θ,DΘ/Dt) is a 7×1 vector of centrifugal and Coriolis terms, G(Θ) is a 7×1 vector of gravity terms.

The kinematic model inputs are IMU, gyroscope, magnetic flux valve (3 axis compass), gravity, linear acceleration, and rotation vector sensors. Android platform sensors provides augmented data based on standard unites of measure, for each Cartesian axis (XYZ) in m/s² for any acceleration, gravity, in rad/s for gyroscope, and unit-less for rotation vector can be done. In this manner, simple vector addition, subtraction, multiplied by rotation vector. This allow to indicate a 3D pattern of acceleration vector. Integrating ones will provide pattern of velocity diagram, integrating twice will provide pattern of movement let us assume a stand still position. This position can be any position, to which current position is related. As a non-limiting example, assuming charging point of the device as baseline position, on which gravity is measured stable, with linear acceleration input, rotation vector, gyro input are equals 0, then any other measurement is related to this position, on Polar or Cartesian axis. Such a point may also be triggered when the health care worker is entering a place such as bedside, a room and the like, in which an operation such as hand hygiene, environmental cleaning, training, PPE usage, gloving, is to be performed. Any transformation can be realized using Euler angles or other transformation known in the field.

Hand hygiene processes such as Handwash or hand rub are based on several reciprocating movements of relatively small magnitude (easily detected with a change of acceleration direction), which are required to be performed several times, where gloving or personal protective equipment placements are described with different patterns. Close proximity sensor can be realized using a proximity sensor available from Omron, Japan, as a simple RLC sensor, PIR, Thermopile, etc. in addition, to using a PIR, when washing hands, a change in the average temp is expected, hence validating a watery compound is used with Handwash.

Typically, patterns of the hand movement do not exactly repeat themselves over time, similar to personal signature.

Therefore, a weighted 3D simple configuration, such as a ball, or an egg is derived. Dimension of these configurations are the allowed margin of error. This allows margin of error is either factory set, or defined for each user during the training stage. For ease of explanation, FIG. 11 shows a schematic simplified model in 2D configuration such as a circle or an ellipse positioned in space is described, using Cartesian coordinate system (XYZ) modified when necessary with Euler angles, as a no limiting example, opening of bed-side curtains, as described below.

Arced lines describe position of the hand, where straight lines describe movement of hand relative to the shoulder. Same operation is cleaning of surfaces first sweep.

For any timeframe, a position is calculated as a circle, summing XYZ, provides line of motion as can be seen, a movement pattern can be derived by summarizing weight of a circle to a 2D or a 3D line. Assuming an ellipse, where distance of centers of gravity is the linear acceleration, relative, as a no limiting example, to gravity, yields different operation. The ellipse distance of centers is different when opening a curtain and cleaning of surface. Adding to this the change of direction of line of motion, and number of times operated, yields cleaning of surface.

In another embodiment, the entire hand motion detection system and HCW actions is done through a pair of goggles equipped with a camera, a data display, a set of indicator lights, communication capabilities, etc. The personal goggle may be used instead of PCMB device 200 or in addition to PCMB device 200.

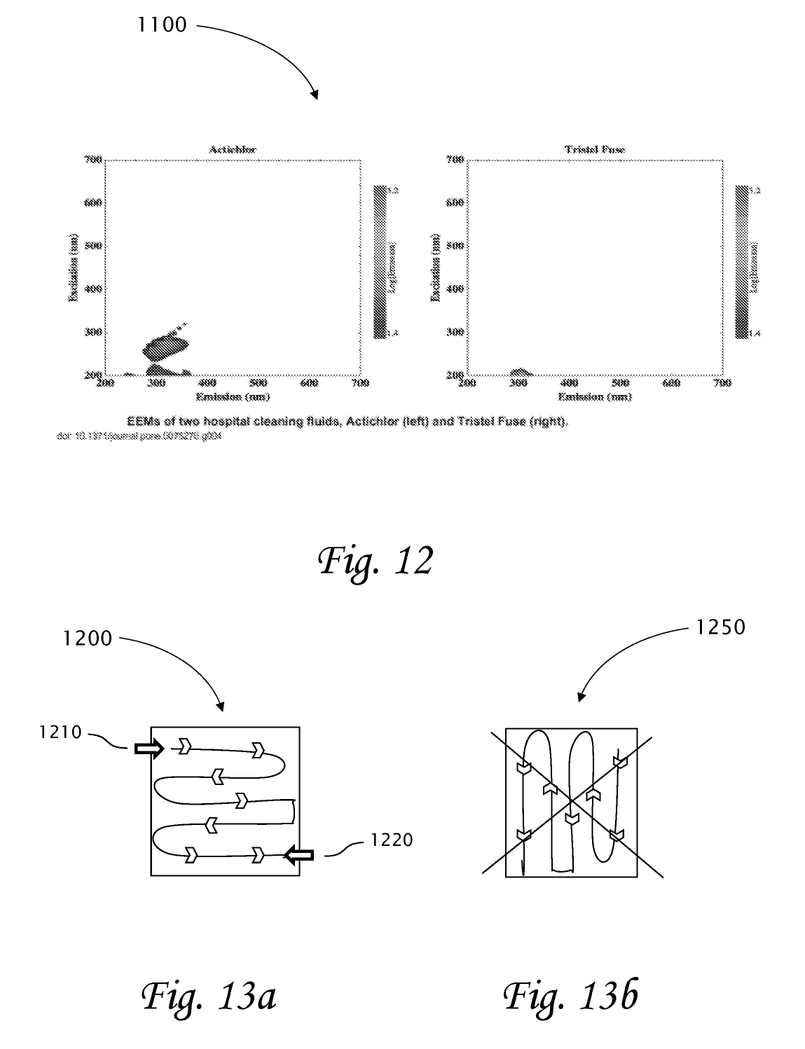

The goggle is equipped with a camera having an out of focus camera to capture a surface to be cleaned/disinfected. The camera is configured to detect the edge of an area that needs to be treated and follows the hand motions on the treated surfaces, to ensure full coverage with the used cleaner disinfectant. The goggle is further equipped with an UV emitting light source, such as led light, with variable excitation wavelengths in the range of 200-300 nm, wherein the camera is configured to capture the emitted fluorescence images of the surface fluorescence that results from the application of the used cleaning disinfecting products, and a to display and/or a visual indicator to indicate if the process was completed and satisfy the required standard. The range of emission captured for NaDCC as well as for Chlorine Dioxide, is 250-350 nm, with a pick at 295 for both, as shown in diagrams 1100 of FIG. 12. The goggle includes a BT communication capability with PCMB device 200, to have the images and motions analyzed in real time and to transmit the collected data to server/cloud 80 data logs 156 for further processing and analysis. As infection-control-practices-monitoring system 100 identifies that the cleaning/disinfection process was performed correctly, from point of view hand motions, that is in the correct manner and direction (detected by the hand motion monitoring), the surface was completely covered with the disinfectant solution (detected by the UV cam), and the contact time was according to the product use instructions, the HCW receives an indication on PCMB device 200 or the goggles (that is equipped a display and/or visual indicator) informing him/her if the process was completed to the required standard. it should be noted that the above 2 different disinfectants were presented with no limitation, and other disinfectants may be used, wherein adjustments to the proper wave lengths for any other disinfectant can be used to and adopted by infection-control-practices-monitoring system 100.

### Monitoring a Cleaning and Disinfection Process

The performance of cleaning and disinfection process of an area, and hand motion monitoring, is as follows:

The hand motion detection will be done by using smart-wrist-wearable device 300 that contains an accelerometer gyroscope and additional sensors as required. The hand motions detection algorithm will analyze the hand motions performed by the HCW in accordance to the recommended process that is a motion from the cleanest zone to the dirtiest zone with motions from right to left or from left to right as illustrated in charts 1200 and 1250 of FIG. 13a. Motion should start at the distant/top end (1210) of a surface, an area presumed clean as it is less touched by the patient Motion should terminate at the close/low end (1220) of a surface, an area presumed dirty as it is more likely to be frequently touched by the patient.

During the hand motion monitoring of a cleaning and disinfection action, no other path of hand motion is allowed. In an improper way of cleaning and disinfecting a surface, the wipe picks up bacteria on a dirty part of the surface and smears such dirt on the clean part, during the process.

If an improper hand motion is detected such as shown in the non-limiting example shown in FIG. 13b, infection-control-practices-monitoring system 100 provides a warning indication, and a 2^{nd} round of disinfection of the same surface, in the correct path of motions, is expected. If control-practices-monitoring system 100 does not approve the detected the cleaning and disinfection of the specific surface, infection-control-practices-monitoring system 100 provides a failed indication.

Reference is now also made to FIG. 14, showing a schematic flow chart of an exemplary cleaning-and-disinfecting process 1300 for determining that a HCW performed the cleaning-and-disinfecting process properly, according to embodiments of the present invention. It is made clear that the provided embodiments may include only parts of this scheme. Process 1300 uses of smart-wrist-wearable device 300, PCMB 200 and/or the goggle for surface disinfection quality monitoring. Process 1300 is described the cleaning steps (1310) taken by the HCW, coupled by the corresponding monitoring action (1350) performed by Infection-control-practices-monitoring system 100. Process 1300 proceeds as follows:
Step 1352: HCW: Identify the product.
   The HCW identifies the product used by stock container RFID tag or chosen from a menu on a display, in order to choose parameters for the process, according to the particular disinfectant usage instructions.
Step 1312: SYSTEM: PCMB receives the RFID signal, or displays a menu.
   PCMB device 200 receives the RFID signal, or displays a menu.
Step 1354: HCW: Face the target surface.
   The HCW faces the object to be cleaned/disinfected to thereby allow infection-control-practices-monitoring system 100 to identify the object and obtain the surface size data and the number of wipes change/replenish disinfectant required during the disinfecting process.
Step 1314: SYSTEM: PCMB.
   If did not receive an RFID, PCMB imaging device 220 acquires at least one image of the target surface. Infection-control-practices-monitoring system 100 obtains object image identifies the object using the DB, and obtains the surface size and other relevant data.
Step 1356: HCW: Provide a success indication and report.
   The HCW takes a wipe loaded with disinfectant solution using the hand wearing smart-wrist-wearable device 300.
Step 1316: SYSTEM: Hand motion detection.
   Infection-control-practices-monitoring system 100 starts Hand motion detection and wipes count/replenish start/update.
Step 1358: HCW: start wiping.
   The HCW approaches the object to be cleaned/disinfected and start wiping it from the clean side to the dirty side.
Step 1318: SYSTEM: start imaging.
   PCMB imaging device 220 or imaging device of the goggle device is activated in a surface coverage mode with hand motion detection, UV light turned on. Images are captured for UV emission detection and surface coverage validation.
Step 1360: HCW: Provide a success indication and report.
   The HCW pays attention to the indicators and display for guidance & instructions.
Step 1320: SYSTEM: derive surface coverage.
   Infection-control-practices-monitoring system 100 calculates surface coverage derived from the hand motions and the images acquired by the UV sensitive camera.
Step 1325: check if need to change wipe.
   Infection-control-practices-monitoring system 100 determined if a wipe needs to be changed, according to pre-set rules adapted to the wipe and solution used.
   If a wipe was to be change and changed, go to step 1316.
Step 1335: check if done before a change of wipe.
   Infection-control-practices-monitoring system 100 determined if all of the target surface was covered but no n wipes were changed.
   If all of the target surface was covered but no wipes were changed, report failure (Step 1332) to data logs 156 and go to step 1310.
Step 1345: check if the required contact time passed.
   Infection-control-practices-monitoring system 100 determined if the required minimal contact time has passed.
   If the required minimal contact time did not pass, report failure (Step 1342) to data logs 156 and go to step 1310.
   report success (Step 1344) and Exit.
[end of process 1300]

Isolation Training

We have previously described training and monitoring of the performance of some of the required steps and actions, hand hygiene and gloving. In the current invention we are describing the way that the same technology used for those previously described actions can be applied to the different isolation requirements.

The system is based on the use of a RFID tag placed on the box/container in which the different items are kept, a smart-wrist-wearable device 300 to capture the hand motions performed during the process and a personal communication and the PCMB device 200 used for data collection monitoring certain steps analysis and communication.

The PCMB device 200 that is connected to the hospital information system 80 is triggered to obtain isolation and precautions required as the care giver approaches the room he needs to attend. The PCMB device 200 is triggered into anticipating certain activities performed by the care giver prior to entering the room if the data obtained from the hospital indicates a certain isolation protocol required for the patients in that room. A second way to trigger the PCMB device 200 into monitoring isolation requirements is based on the current practice that includes the placement of a sign at the entrance to the room or on a specific patient bed that indicates the type of isolation required. By incorporating a specific RFID tag into these Isolation signs, the PCMB device 200, as previously described, equipped with RFID detection ability is triggered by the data obtained from the RFID equipped sign is triggered into the proper isolation protocol and anticipates the performance of a specific set of activities that requires a specific sequence of hand motions as described in the following tables as detailed for each of the actions. The care giver is provided a signal visual or auditory of the isolation requirements and the PCMB device 200 monitors its performance. When the required steps are completed in the right way the care giver is provided with a signal that the procedure was completed and that he is ready to enter the room or approach the patient. If the procedure was not performed in accordance with the requirements the HCW is provided with a signal visual or auditory that the procedure was not performed correctly. The procedure monitoring data and results are then sent to the hospital server DB 152 for performance data collection and analysis.

A major part of infection control activities includes isolations of patients for his protection or for protection of other patients. For this purpose and for other purposes in the HCW daily routine there is a need for using personal protective equipment—PPE. The PPE includes, gloves, goggles, face mask, hair cover, splash protector, shoe covers etc. for different isolation conditions there are different requirements for PPE to be used as can be seen from the following:

Standard precautions: are the minimum infection prevention practices that should be used in the care of all patients all of the time. These practices are designed to both protect the healthcare worker and to prevent the healthcare worker from spreading infections among patients.

Standard Precautions Include:
1. Hand hygiene
2. Use of personal protective equipment (e.g., gloves, gowns, masks)
3. Safe injection practices
4. Safe handling of potentially contaminated equipment or surfaces in the patient environment, and
5. Respiratory hygiene/cough etiquette.

There are Three different Types of Specific Isolation Precautions:
1. Contact Isolation Precautions—used for infections, diseases, or germs that are spread by touching the patient or items in the room (examples: MRSA, VRE, diarrheal illnesses, open wounds, RSV).
2. Droplet Isolation Precautions—used for diseases or germs that are spread in tiny droplets caused by coughing and sneezing (examples: pneumonia, influenza, whooping cough, bacterial meningitis).
3. Airborne Isolation Precautions—used for diseases or very small germs that are spread through the air from one person to another (examples: tuberculosis, measles, chickenpox).

Contact Isolation Precautions—a HCW should:
Wear a gown and gloves while in the patient's room.
Remove the gown and gloves before leaving the room.
Clean hands (hand washing or use hand sanitizer) when entering and leaving the room.
Visitors must check with the nurse before taking anything into or out of the room.

Droplet Isolation Precautions—Healthcare Workers should:
Wear a surgical mask while in the room. Mask must be discarded in trash after leaving the room.
Clean hands (hand washing or use hand sanitizer) when they enter the room and when they leave the room.

Airborne Isolation Precautions—Healthcare Workers should:
Ensure patient is placed in an appropriate negative air pressure room (a room where the air is gently sucked outside the building) with the door shut.
Wear a fit-tested NIOSH-approved N-95 or higher level respirator while in the room. Mask must be discarded in trash after leaving the room.
Clean hands (hand washing or use hand sanitizer) when they enter the room and when they leave the room.
Ensure the patient wears a surgical mask when leaving the room.
Instruct visitors to wear a mask while in the room.

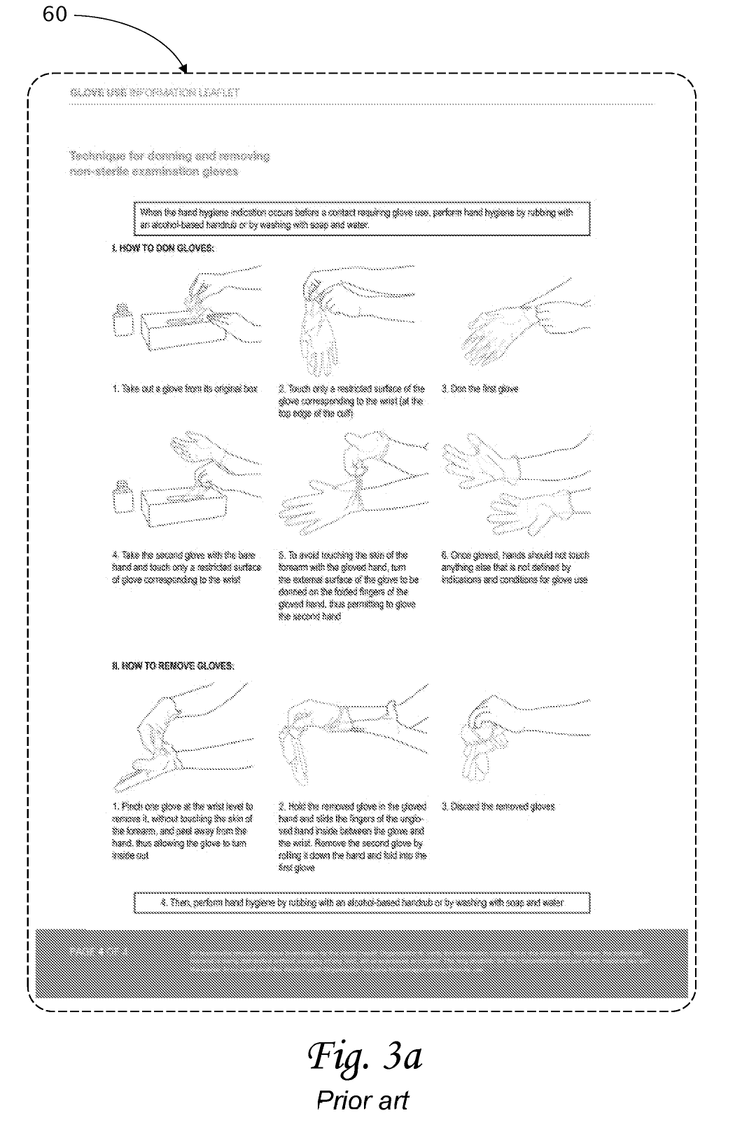

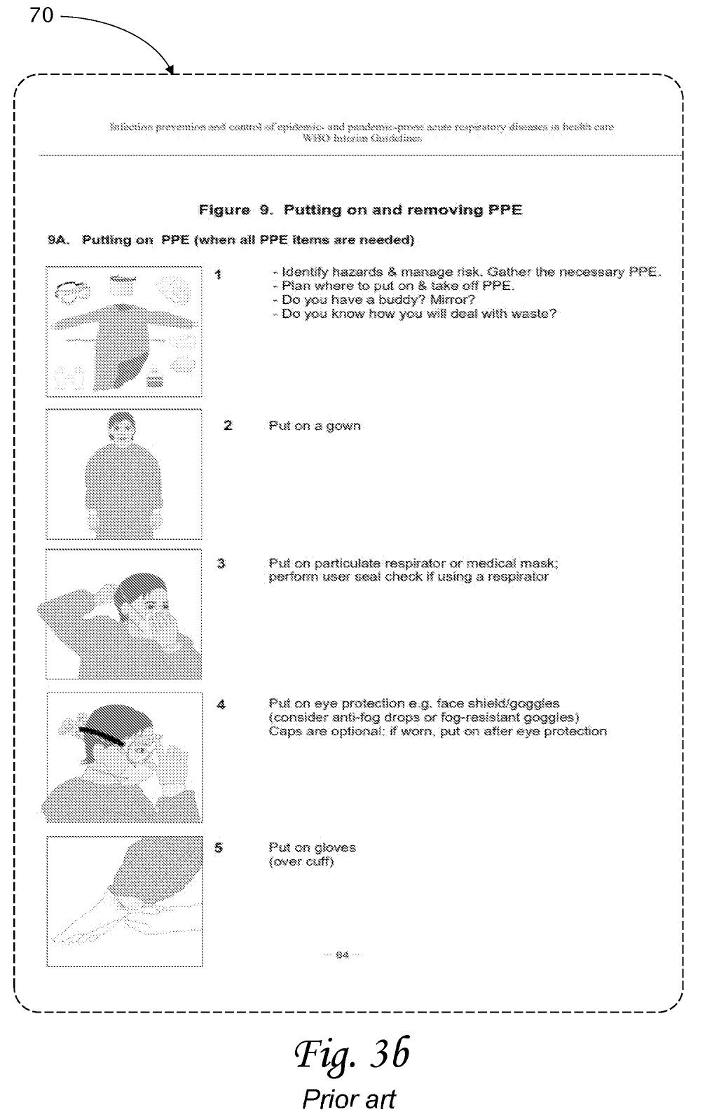

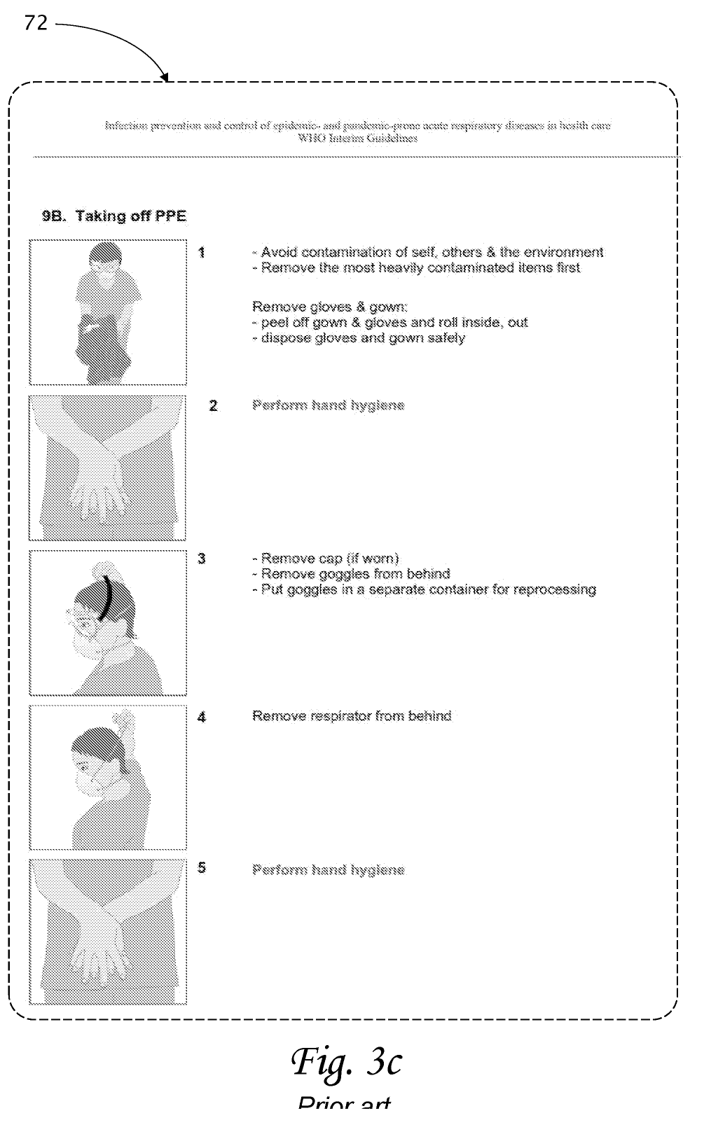

Wearing and removing gloves is one of the aspects of the present invention, being an important part of infection control and referred to as universal precautions. These precautions include hand gloving and hand hygiene in accordance to the risk to the HCW for exposure to blood and body fluids. The requirement for glove wearing (donning) should be performed at certain moments and not all the time. The moments that require glove wearing and the way to do so have been defined in a WHO document and presented in the WHO chart, depicted in FIG. 3a.

Determining when Donning is Indicated and Required

The requirement for donning with non-sterile gloves is related to the risk of exposure of the HCW to blood and body fluids during the performance of the medical procedure. To identify the Moments, different info sources such as patient data base are used with the indicated procedures. The “personal badge for monitoring” (previously described) can be used and monitor the place of the hands of the HCW in relation to the patient's body silhouette, as captured by the IR sensor or another image capturing sensor that is in the “personal badge for monitoring”, or a combination of the patient chart info and the hand positioning.

The System Components include:
A gloves box with a RFID tag on it.
A system that identifies the place where the gloves box is.
A personal monitoring and comm. Badge (PMCB 200).
A hand motion detection bracelet (smart-wrist-wearable device 300)—a hand worn device with various sensors for hand motions detection (e.g. Accelerometer, gyroscope, proxy meter and more) (HMDB).
A data base collection cloud server.
A special algorithm to analyze hand motions and according to the WHO requirements.

Determining the quality of glove donning in accordance to the WHO requirements:

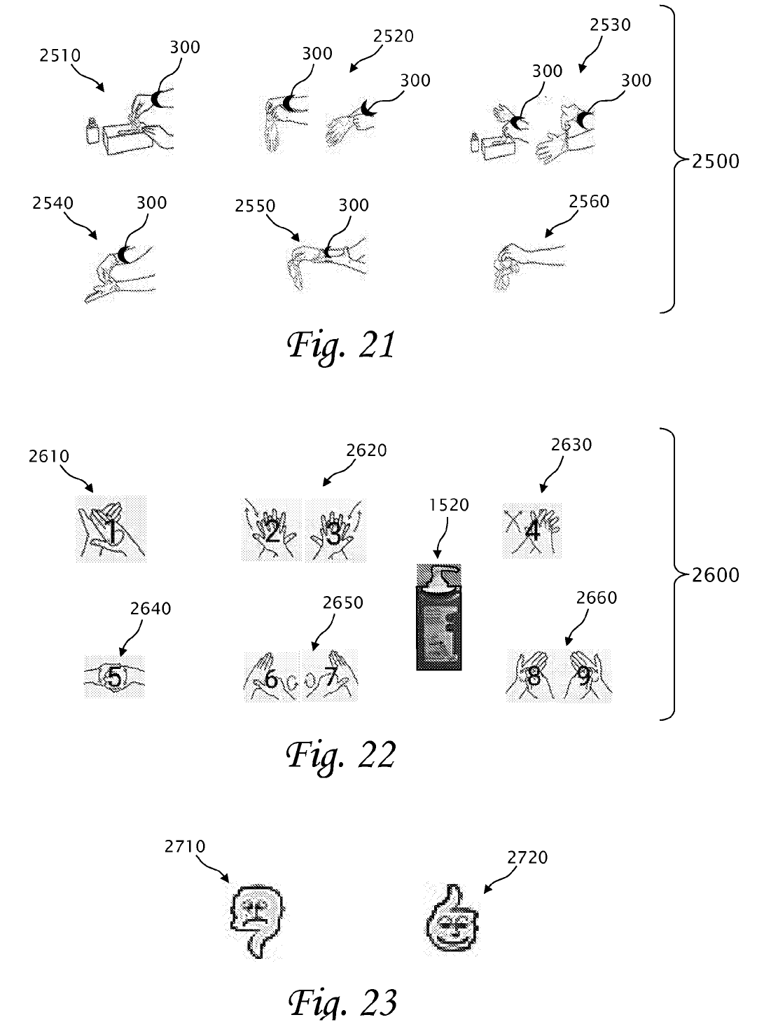

The glove donning is dependent on hand motions that are required to perform a correct glove donning. The hand motions are detected by the hand worn smart-wrist-wearable device 300 that contains a plurality of sensors that are used to determine the hand motions performed. The hand motion data collected is than analyzed by a special algorithm that compares the hand motions performed during glove donning or removing with the hand motions that are according to the WHO process. One of the possible ways to analyze the hand motions is described in the following table 1 and group 2500 of iconic drawings in FIG. 21:

TABLE 1

| | | | |
|---|---|---|---|
| Perform hand | Referring to iconic | Referring to iconic | Referring to iconic |
| hygiene if | drawing 2510. | drawing 2520. | drawing 2530. |
| indicated. | The PMCB detects | From a horizontal | With the hand with |
| Action monitored | the RFID on the | gyro resting | the HMDB in a |
| and indicated in | gloves box as the | position a | stationary position |
| accordance with | HCW approaches | backward motion | to then a short |
| WHO 5 Moments | the POC and | of pooling towards | move forward to a |
| | triggers the PMCB | the HCW body. | horizontal gyro |
| | into hand motion | And then a vertical | position to grab the |
| | monitoring mode. | gyro and | 2^{nd} glove and a |
| | The HMDB starts | downwards motion | backward long |
| | to collect hand | with a rotating | motion to pool the |
| | motions data are | forward motion. | glove over the |
| | compared to | | other hand. |
| | motions required | | |
| | to complete the | | |
| | glove donning. 1^{st} | | |
| | motion (by using | | |
| | the hand with the | | |
| | HMDB on it) from | | |
| | a horizontal hand | | |
| | gyro position a | | |
| | sharp back pooling | | |
| | of the hand motion | | |
| Glove removing | Referring to iconic | Referring to iconic | Referring to iconic |
| | drawing 2540. | drawing 2550. | drawing 2560. |
| | The hand with the | The hand with the | The gloves are |
| | HMDB is in a | HMDB moves to a | discarded. |
| | horizontal gyro | vertical position | |
| | position and | and performs a | |
| | performs along | long and slow | |
| | motion forward to | motion backward | |
| | remove the glove | to remove the | |
| | of the other hand. | second glove | |
| A hand hygiene is | | | |
| performed | | | |
| Action monitored | | | |
| and indicated in | | | |
| accordance with WHO | | | |
| 5 Moments | | | |

The above described glove donning hand motion monitoring and interpretation is one of other methods of detection of hand activity it can be done by video capturing and analysis of the motions captured or an algorithm that analyzes the hand motion based on gesture analysis based on neuroscience.

Wearing gloves or PPE, yields different signature of the line of circles, hence detecting hand motion for hand hygiene, PPE environmental cleaning, gloving.

The following table describes the Personal Protective Equipment (PPE) monitored hand motions in accordance to the WHO guidelines tables:

The correct sequence of performance is determined by the sequence of the HCW approaching the various containers/ boxes of PPE and gathering the necessary items according to the type of isolation or precautions. PCMB device 200 determines if the correct items were collected by identifying the RFID on the container/box. After the items were collected the detection algorithm is triggered into motion detection mode specific for the PPE items chosen by the HCW and the anticipated sequence is then monitored according to the specific isolation protocol requirements, as previously identified based on the RFID incorporated in the isolation sign.

Step 1—prior to any isolation PPE wearing sequence the system anticipates the performance of hand hygiene protocol and monitors it.

Step 2—if a gown is required: putting on the gown. The hand motion sequence is initiated by the insertion of the hand with smart-wrist-wearable device 300 first into the gowns sleeve this motion is a long forward motion of the arm then a backward and to horizontal position motion of the same arm to hold the other side of the gown and assist in inserting the second hand into the second sleeve. The next motion is a backwards and downwards motion of the hand with smart-wrist-wearable device 300 to tie the ribbons in the back of the gown the process is validated with the performance of several short hand motions corresponding to tying motions

Step 3—if a facial mask is required: Putting on a mask. Depending of the type of mask used

1) If a facial mask with an elastic ribbon (usually in respiratory isolation) is used then the mask is placed in the palm of the arm without smart-wrist-wearable device 300 and mask is lifted to the HCW face the hand with smart-wrist-wearable device 300, then performs an upward motion and then a backward and downward motion of pulling back the elastic over the top of the head. Then the arm with smart-wrist-wearable device 300 is brought to the front of the face to perform seal checking.

2) If a facial mask with tying ribbons is used (usually in all other isolations) then the motions detected include a backward and upward motion of the hand with smart-wrist-wearable device 300 on and then a tying short motion. After that a short downward motion followed with a tying motion and then the return of the hand to its natural position.

Step 4—if goggles or face shield are indicated: the expected hand motion would be an upward hand motion of the hand with smart-wrist-wearable device 300 that is holding the goggles. If the goggle has an elastic ribbon, then the HCW is instructed to use the hand with smart-wrist-wearable device 300 on to pull the ribbon over the head. The expected hand motions are the hand with smart-wrist-wearable device 300, then performs an upward motion and then a backward and downward motion of pulling back the elastic over the top of the head. If a cap is required, it should always be worn after putting on the goggles. If the cap is an elastic ribbon type, then the expected hand motion would be an upward and outwards motion of the hand with smart-wrist-wearable device 300 on and then a downward motion. If the cap is a ribbon type cap the expected hand motions are: the hand with smart-wrist-wearable device 300 on moves upwards and outwards and then downwards. The next hand motions are short hand motions of tying.

Step 5—Putting on gloves. Prior to putting on the gloves hand hygiene is required then the gloves are put on. Both hand motions monitoring processes have been described previously.

As both actions are completed the guidelines require the gloves to be pulled over the sleeves the last motion of the gloving should be a long backward puling of the hand with smart-wrist-wearable device 300 on.

As the process is completed in the correct sequence and the right motions the HCW is provided a signal visual or auditory to indicate the completion. The system will have an override button that will enable the HCW to override the monitoring in emergency situations each override use will have to be justified later by the clinical emergency so it will not be abused.

PPE removal process in accordance with the WHO recommended process, monitoring by hand motion detection.

As the HCW finishes the required activity and is about to exit the room or move to the next patient the used PPE must be removed in a sequence described in the WHO guidelines as shown in the protocol above in order to prevent contamination of the HCW the environment and the next attended patient.

The following process monitored by the hands motions should be performed:

Step 1—Start PPE removal with the hand smart-wrist-wearable device 300 is on it with a backward motion to untie the knot of the gown ribbons in the back. The hands perform as set of short motions to untie the ribbons. The next motion is a forward and upwards diagonal motion of the hand with smart-wrist-wearable device 300 on to the opposite shoulder. The next motion is a long pulling downward motion the hand then moves back to its basic position and then pulled back with a long backward motion with some additional short hand motions to assist the glove removal.

Step 2—Perform a hand hygiene the system will monitor this stage as we described earlier.

Step 3 —Cap removal and goggles: using the hand with smart-wrist-wearable device 300 on it pool the cap (if it is an elastic ribbon type) off the head the hand motions include an upward and backward motion and then a forward and down ward motion while removing the cap. If the cap is a ribbon type cap, then the hand motions would be an upward and backward motion and then a set of short motions while untying the ribbons then a forward and downwards hand motion while pooling the cap off the head. The next motions detected are connected to the goggles removal depending on the type of goggles. If it is an elastic ribbon type, then the hand motions would be an upward and backward hand motion and then a forward and downward hand motion while pulling the goggles off over the head. If it is an eye glass type goggles, then they should be removed using the hand with smart-wrist-wearable device 300 on it the hand motion detected would be an upward hand motion and then a forward and downward motion.

Step 4—Face mask removal: depending on the type of mask used the following hand motions are expected. If the face mask is with an elastic ribbon, then the expected hand motions are: an upward and back ward motion of the hand with smart-wrist-wearable device 300 on it and then a forward and downward motion to remove the face mask over the head. If the mask is a ribbon type, then the expected hand motions are: an upward hand motion of the hand with smart-wrist-wearable device 300 on it and then some short hand motions while untying the ribbons knot and then a downward motion while removing the mask.

Step 5—Perform hand hygiene that will be monitored as we described previously.

The hand motions collected by smart-wrist-wearable device 300 are transmitted in real time to the PCMB device 200 and are analyzed so corrective guidance can be provided in real time to the HCW, the data is transmitted to the hospital server and analyzed for the purposes of monitoring compliance by the infection control team.

The system will be able to identify the type of face mask used, the type of cap being used and the type of goggles used according to the details received by the system from the RFID tag that was on the box/container the items were collected from and the proper hand motion detection algorithm will be utilized for the process monitored.

Sensors

Accelerometer—Measures the acceleration force in m/s2 that is applied to a device on all three physical Cartesian axes (x, y, and z), including the force of gravity.

Ambient temperature—Measures the ambient room temperature in degrees Celsius (° C.)

Gravity—Measures the force of gravity in m/s2 that is applied to a device on all three physical axes (x, y, z).

Gyroscope—Measures a device's rate of rotation in rad/s around each of the three physical axes (x, y, and z).

Proximity—Measures the proximity to second hand.

Linear acceleration—Measures the acceleration force in m/s2that is applied to a device on all three physical axes (x, y, and z), excluding the force of gravity.

Magnetic field—Measures the ambient geomagnetic field for all three physical axes (x, y, z) in μT.

Orientation—Measures degrees of rotation that a device makes around all three physical axes (x, y, z). May obtain the inclination matrix and rotation matrix for a device by using the gravity sensor and the geomagnetic field sensor

2 hand proximity PIR—Measures distant temperature of an object, less the 50 cm.

2 hand Proximity RLC13 Measures the relative proximity I change of RLC impedance

Proximity—Measures the proximity of an object in cm relative to the device, for example an ultrasound proximity sensor. This sensor is typically, with no limitations, used to determine distance to an object that is less than 10 cm away. Together with PIR, one can differentiate distance to an object or live tissue.

Rotation—Measures the orientation of a device by providing the three elements of the device's rotation vector.

For each 3D sensor, accelerometer, magnetic flux valve:
Averaged variance over 3 axes
RMS of signal derivative.
Mean of signal derivative.
Average entropy over 3 axes.
Averaged cross correlation between each 2 axes.
Average range over 3 axes.
Average main frequency of the FFT over 3 axes.
Total signal energy averaged over 3 axes.
Energy of 0.2 Hz window around the main frequency over total FFT energy (3 axes average).
Averaged skew over 3 axes.
Averaged range of cross covariance between each 2 axes.
Averaged mean of cross covariance between each 2 axes.
   1) FIRST Feature Selection: There are 2 types of margins that are used in machine learning to define classifier confidence when making a decision. The first is the distance margin which looks at maximizing the distance between an instance and the decision boundaries, and the second is the machine stored patterns, factory set or recorded during training process margin which is the distance between the machine stored patterns, factory set or recorded during training process and the closest machine stored patterns, factory set or recorded during training process that assigns an alternative label to the given instance. The FIRST algorithm for feature selection [2] is an iterative algorithm that utilizes machine stored patterns, factory set or recorded during training process margins to assign weights to features in order to increase the margin between samples in different classes. The following update rule is used per iteration:

w₁=w₁+(xᵢ−nearmiss(x)ᵢ) 2−(xᵢ−nearhit(x)ᵢ) 2 (1)

In equation 1, wᵢ refers to weights per feature i, xᵢ is the value of the instance for nearhit(xᵢ) and nearmiss(xᵢ) refer to the nearest point to xᵢ with the same and different labels respectively.
FIRST has been used extensively in literature due to its speed and simplicity in weighting relevant features. However, it does not have mechanisms for eliminating redundant features.
2) Simba Feature Selection: The Simba (Iterative Search Margin Based Algorithm) for feature selection is similar to FIRST in terms of updating feature weights to provide maximum margins. However, unlike FIRST, Simba performs a gradient ascent over weights to re-evaluate distances according to the weight vector w. This allows it to cope better with redundant features. Correlated features could be chosen by Simba if they contribute to overall performance.
3) mRMR (minimum Redundancy Maximum Relevance) Feature Selection: The mRMR framework for feature selection [3] aims to find features that provide the maximum relevance (equivalent to maximum dependency between features and class labels) as well as the minimum redundancy. These two criteria are combined in an incremental selection scheme using mutual information to assess relevance and redundancy. Mutual information between two random variables x and y can be defined in terms of their probabilistic density functions p(x) and p(y) as well as their joint probability p(x, y):

I(x, y)=∫∫p(x, y)log p(x, y) p(x)p(y) dₓd_{y} (2)

Incremental search methods are used to find feature sets (S) that satisfy the mRMR operator Φ(D, R)=D−R where D and R are the relevance (approximating dependency) and redundancy respectively. Features that satisfy both of the following criteria are selected (c is the class label and xi the feature):

$$\begin{matrix}max D ( S , c ) , D = \frac{1}{\vert S \vert} {\sum }_{{x}_{i} ∈ S} I ( {x}_{i} , c ) & ( 3 ) \\ min R ( S ) , R = \frac{1}{{\vert S \vert}^{2}} {\sum }_{{x}_{i} , {x}_{j} ∈ S} I ( {x}_{i} , {x}_{j} ) & ( 4 )\end{matrix}$$

C. Classification

We opted for classifiers known for their speed as the datasets were relatively large when all subjects were combined. For this reason, the knn classifier (K-nearest neighbor) is used with different values of k to assess the effect of outlier points. A Bayesian Classifier is also used where Gaussian distributions were used to model the priors of classes and the posterior probability of a point x belonging to a class (Ck) calculated as:

P(Cₖ|x)=aP(x|Cₖ)P(Cₖ) (5)

The normalizing constant a, is expressed as follows for a total number of classes K:

$$α = \frac{1}{{\sum }_{k = 1}^{K} P ( x \vert {C}_{k} ) P ( {C}_{k} )} \qquad ( 6 )$$

An additional feature that can be incorporated in to infection-control-practices-monitoring system 100 includes electronic medical equipment such as, with no limitations, respirators, IV pumps, feeding pumps dialysis machines, hospital beds but not limited to them an electronic component that will communicate with the CG PCMB device 200 and will require a signal that validates the performance of proper hand hygiene prior to approaching the patient or the device. Communication may use IOT. If hand hygiene was not performed or not properly performed, the device produces a warning signal that will end only after the HCW has performed the hand hygiene properly before approaching the patient. The event will be reported to the central DB.

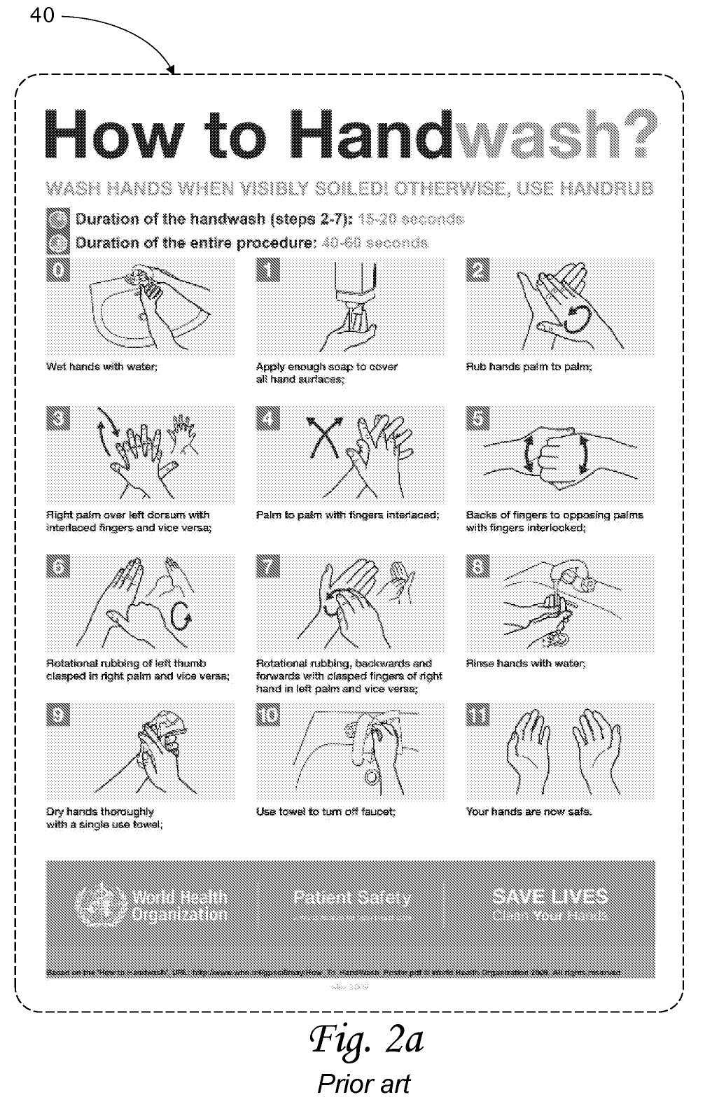

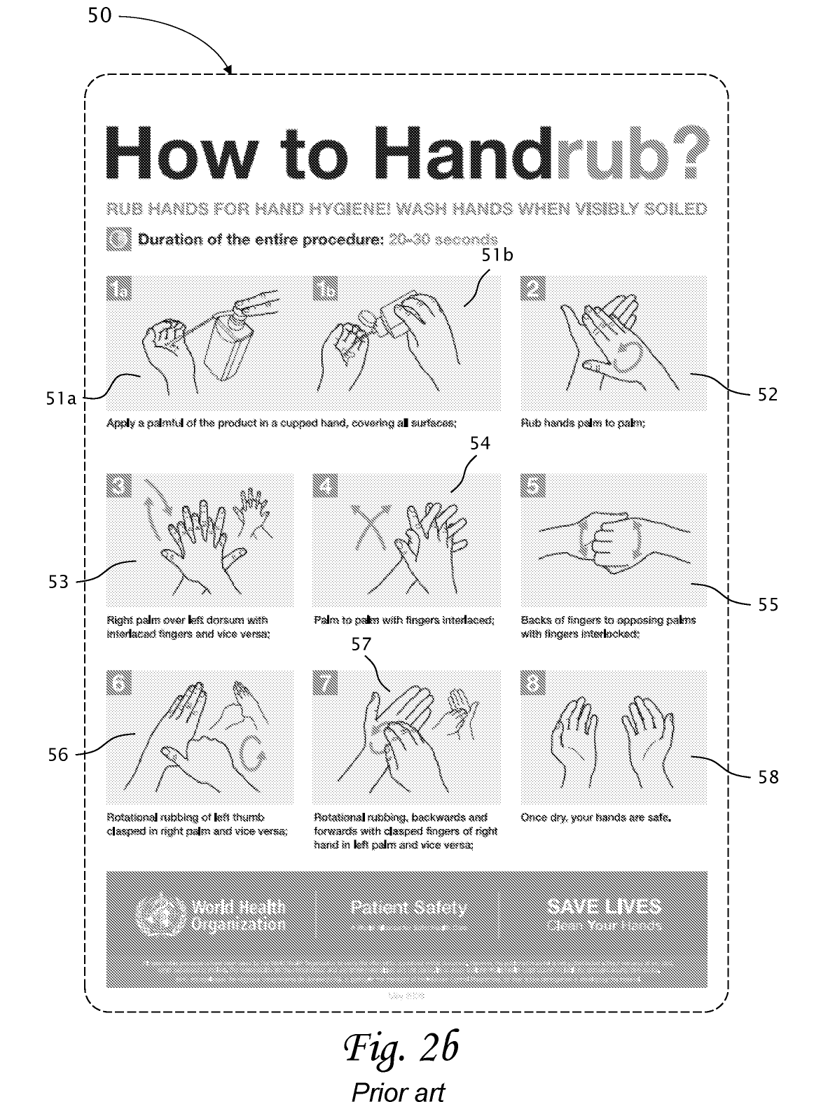

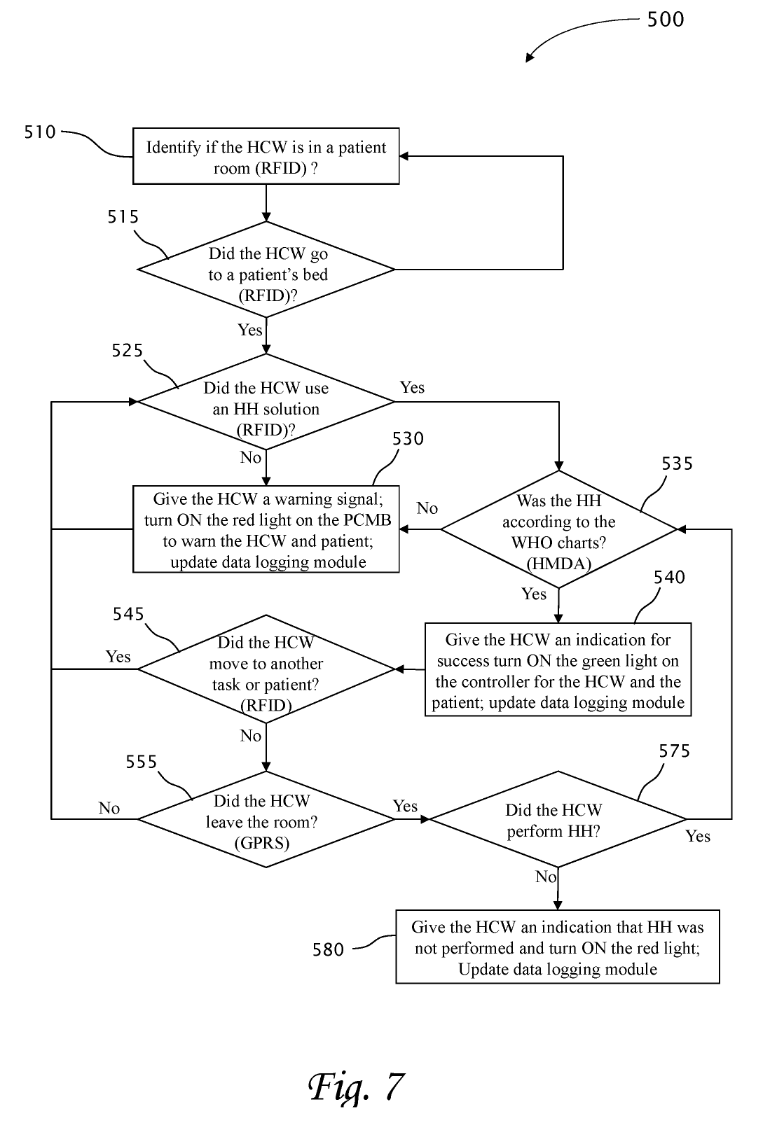

Reference is now also made to FIG. 7, showing a schematic flow chart of an exemplary HH-monitoring process 500 for determining a HCW performed the WHO hand hygiene procedures properly, according to embodiments of the present invention. It is made clear that the provided embodiments may include only parts of this scheme. Process 500 starts monitoring a HCW in step 501, when the HCW enters the room of a patient. Process 500 proceeds as follows:
Step 510: Identifying the patient's room.
   Infection-control-practices-monitoring system 100 determines the HCW location using, for example, RFID reader 240 to read the RFID tag of the patient's room.
   It should be noted other positioning methods known in the art may be used to determine the positioning of the HCW. For example, using GPRS to determine the position of the smart mobile phone of the HCW. In another method, the position of the HCW is be determined using a Wi-Fi positioning scheme.
Step 520: check if the HCW performed the Hand Hygiene (HH).
   Optionally, Infection-control-practices-monitoring system 100 determines if the HCW has performed the HH in accordance to the WHO procedures, as shown in FIGS. 2a and 2b.
   To determine if the HCW has performed the HH in accordance to the WHO procedures, infection-control-practices-monitoring system 100 performs the steps outlined in FIG. 7.
   If infection-control-practices-monitoring system 100 determines if the HCW did not performed the HH in accordance to the WHO procedures, go to step 530.
Step 525: check if the HCW has used an HH solution.
   Infection-control-practices-monitoring system 100 determines if the HCW has used an HH solution.
   According to the present invention, each bottle 120 that contain the solution is labeled with a unique RFID tag 122. Thereby, when the HCW approaches, RFID reader 240 of the HCW PCMB 200 reads RFID tag 122. When the HCW uses bottle 120 it is done by predictable hand motions. The HCW PCMB 200, using the sensors and the sensors, such as accelerometer 322, detects the performed non-Hand-Hygiene hand motion and matches the detected non-Hand-Hygiene hand motion is matched against a database of non-Hand-Hygiene hand motions. Thereby, infection-control-practices-monitoring system 100 determines if the HCW has used an HH solution.
   If infection-control-practices-monitoring system 100 determines if the HCW used an HH solution, go to step 535.
Step 530: Provide warning indication and report.
   Infection-control-practices-monitoring system 100 has determined that a required WHO HH required procedure, at a given WHO Moment, has not been performed. Therefore, infection-control-practices-monitoring system 100 issues a warning to the HCW, for example and audible warning. Furthermore, infection-control-practices-monitoring system 100 turns ON red light on 260r the PCMB device 200 to warn the HCW and patient, and updates data logging module 280.
   Go to step 525.
Step 535: check if the HCW performed the Hand Hygiene (HH) properly.
   Infection-control-practices-monitoring system 100 determines if the HCW has performed the HH in accordance to the WHO procedures, as shown in FIGS. 2a and 2b.
   In order to determine if the HCW has performed the HH in accordance to the WHO procedures, infection-control-practices-monitoring system 100 performs hand motion analysis, as previously described.
   If infection-control-practices-monitoring system 100 determines if the HCW did not performed the HH in accordance to the WHO procedures, go to step 530.
Step 540: Provide a success indication and report.
   Infection-control-practices-monitoring system 100 has determined that a required WHO HH required procedure, has been performed. Therefore, infection-control-practices-monitoring system 100 issues a success indication to the HCW, for example and audible of vibratory signal. Furthermore, infection-control-practices-monitoring system 100 turns ON green light on 260 g the PCMB to warn the HCW and patient, and (step 550) updates data logging module 280.
Step 545: check if the HCW moved to another patient.
   Infection-control-practices-monitoring system 100 determines if the HCW moved to another patient, for example, by reading the RFID tag 132 of another patient.
   If infection-control-practices-monitoring system 100 has determined the HCW has moved to another patient, go to step 525.
Step 555: check if the HCW left the room.
   Infection-control-practices-monitoring system 100 determines if the HCW has left the room by determining the HCW location, using, for example, RFID reader 240 to read the RFID tag at his/her location.
   It should be noted other positioning methods known in the art may be used to determine the positioning of the HCW. For example, using GPRS to determine the position of the smart mobile phone of the HCW. In another method, the position of the HCW is be determined using a Wi-Fi positioning scheme.
   If infection-control-practices-monitoring system 100 has determined the HCW did not leave the room, go to step 525.
Step 575: check if an additional HH was performed by the HCW prior to patient contact.
   Infection-control-practices-monitoring system 100 determines if an additional HH was performed by the HCW after attending another patient in the same room.
   If infection-control-practices-monitoring system 100 has determined that an additional HH was performed by the HCW after attending another patient in the same room, go to step 535.
Step 580: Provide warning indication and report.
   Infection-control-practices-monitoring system 100 has determined that a required WHO HH required procedure, at a given WHO Moment, has not been performed. Therefore, infection-control-practices-monitoring system 100 issues a warning to the HCW, for example and audible warning. Furthermore, infection-control-practices-monitoring system 100 turns ON red light on 260r the PCMB device 200 to warn the HCW and patient, and updates data logging module 280.
Exit.
[end of process 500]

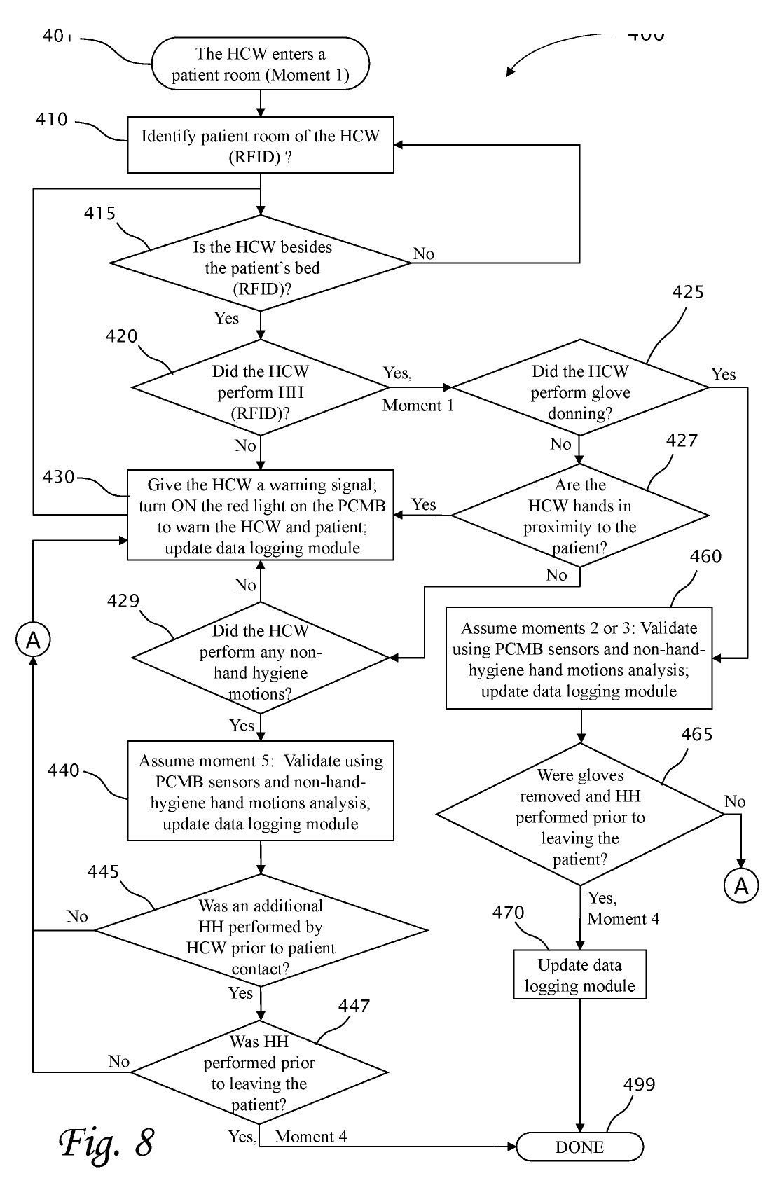

Reference is now also made to FIG. 8, showing a schematic flow chart of an exemplary 5-moments-motion-detection process 400 for identifying all of the WHO “5 moments” that require performing the WHO hand hygiene procedures, according to embodiments of the present invention. It is made clear that the provided embodiments may include only parts of this scheme. Process 400 starts monitoring a HCW in step 401, when the HCW enters the room of a patient. Process 400 proceeds as follows:
Step 410: Identify the patient's room.
   Infection-control-practices-monitoring system 100 determines the HCW location using, for example, RFID reader 240 to read the RFID tag of the patient's room.
   It should be noted other positioning methods known in the art may be used to determine the positioning of the HCW. For example, using GPRS to determine the position of the smart mobile phone of the HCW. In another method, the position of the HCW is be determined using a Wi-Fi positioning scheme.
Step 415: check if the HCW is next to the patient's bed.
   Infection-control-practices-monitoring system 100 determines if the HCW is near the patient bed using, for example, RFID reader 240 to read the RFID tag 132 associated with the patient. RFID tag 132 may be positioned on the wrist strap of the patient, on his/her bed, and the like.
   If the HCW did not go to a patient's bed, go back to step 410.
Step 420: check if the HCW performed the Hand Hygiene (HH).
   Infection-control-practices-monitoring system 100 determines if the HCW has performed the HH in accordance to the WHO procedures, as shown in FIGS. 2a and 2b.
   In order to determine if the HCW has performed the HH in accordance to the WHO procedures, infection-control-practices-monitoring system 100 performs the steps outlined in FIG. 7.
   If infection-control-practices-monitoring system 100 determines if the HCW did not performed the HH in accordance to the WHO procedures, go to step 430.
Step 425: check if the HCW performed the Hand Hygiene (HH).
   Infection-control-practices-monitoring system 100 has determined that the HCW has performed the HH in accordance to the WHO procedures.
   Infection-control-practices-monitoring system 100 determines if the HCW has performed glove donning in accordance to the WHO procedures.
   If infection-control-practices-monitoring system 100 determines if the HCW has performed glove donning in accordance to the WHO procedures, go to step 460.
Step 427: check if the hands of the HCW are in proximity to the patient.
   Infection-control-practices-monitoring system 100 has determined that the HCW has performed glove donning in accordance to the WHO procedures. Infection-control-practices-monitoring system 100 determines if the hands of the HCW are in proximity to the patient using living body proximity sensor 320.
   If infection-control-practices-monitoring system 100 has determined that the hands of the HCW are in proximity to the patient, go to step 430.
   Go to step 430.
Step 429: check if the HCW performed any non-hand hygiene motion.
   If infection-control-practices-monitoring system 100 has determined that the hands of the HCW are not in proximity to the patient. Hence, no glove donning is required.
   Infection-control-practices-monitoring system 100 determines if the HCW has performed any non-hand hygiene motion, for example, hand motion characterizing pulling of the patient's privacy curtain.
   If infection-control-practices-monitoring system 100 has determined that the HCW has performed any non-hand hygiene motion, go to step 440.
Step 430: Provide warning and report.
   Infection-control-practices-monitoring system 100 has determined that a required WHO HH required procedure, at a given WHO Moment, has not been performed. Therefore, infection-control-practices-monitoring system 100 issues a warning to the HCW, for example and audible warning. Furthermore, infection-control-practices-monitoring system 100 turns ON red light on 260r the PCMB to warn the HCW and patient, and updates data logging module 280.
   Go to step 415.
Step 440: Assuming WHO Moment 5.
   Infection-control-practices-monitoring system 100 assumes that a WHO Moment 5 has occurred, which requires performing a WHO HH procedure. Infection-control-practices-monitoring system 100 uses PCMB sensors, such as, with no limitations, IR image sensor and proximity sensor, and non-hand-hygiene hand motions analysis to validate that a WHO Moment 5 has occurred.
   In both cases where validation is either successful or unsuccessful—updates data logging module 280.
Step 445: check if an additional HH was performed by the HCW prior to patient contact.
   Infection-control-practices-monitoring system 100 determines if an additional HEI was performed by the HCW prior to patient contact, as required (Moment 5).
   If infection-control-practices-monitoring system 100 has determined that an additional HH was not performed by the HCW prior to patient contact, go to step 430.
Step 447: check if the HH performed prior to leaving the patient.
   Infection-control-practices-monitoring system 100 determines if the additional HH was performed by the HCW prior to leaving the patient.
   If infection-control-practices-monitoring system 100 has determined that an additional HH was performed by the HCW prior to leaving the patient, a WHO Moment 4 occurrence is assumed—go to step 499.
   Infection-control-practices-monitoring system 100 has determined that an additional HH was performed by the HCW not prior to leaving the patient. Go to step 430.
Step 460: Assuming WHO Moment 2 or Moment 3.
   Infection-control-practices-monitoring system 100 assumes that a WHO Moment 2 or Moment 3 has occurred, which requires performing a WHO HH procedure. Infection-control-practices-monitoring system 100 uses PCMB sensors and non-hand-hygiene hand motions analysis to validate that either a WHO Moment 2 or a WHO Moment 3 has occurred.
   In both cases where validation is either successful or unsuccessful—updates data logging module 280.
Step 465: check if the gloves were removed and HH was performed, prior to leaving the patient.
   Infection-control-practices-monitoring system 100 determines if the gloves were removed and HH was performed prior to leaving the patient, as required (either Moment 2 or 3).
   If infection-control-practices-monitoring system 100 has determined that either the gloves were not removed and/or HH was not performed prior to leaving the patient, go to step 430.
Step 470: Assuming WHO Moment 4.
   If there were several improvements in the recognition algorithm, new version of the application or device is published in the cloud for future updates of the software part of the system.
Step 499: Exit.
[end of process 400]

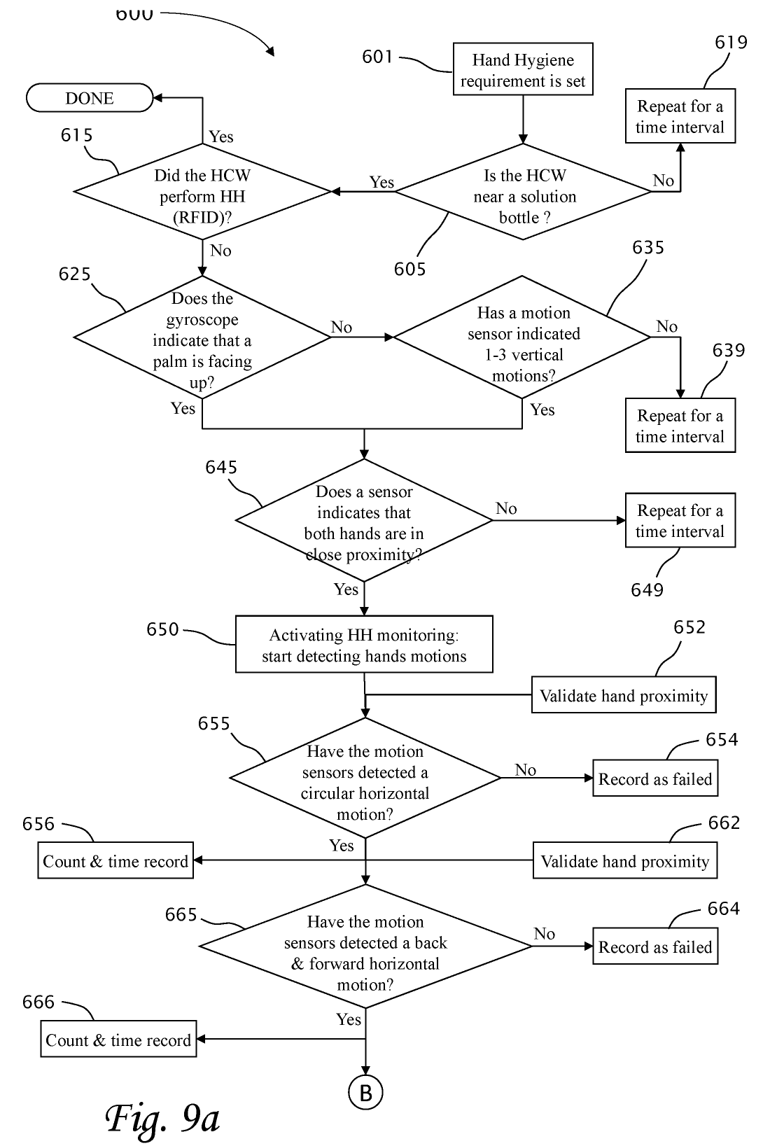

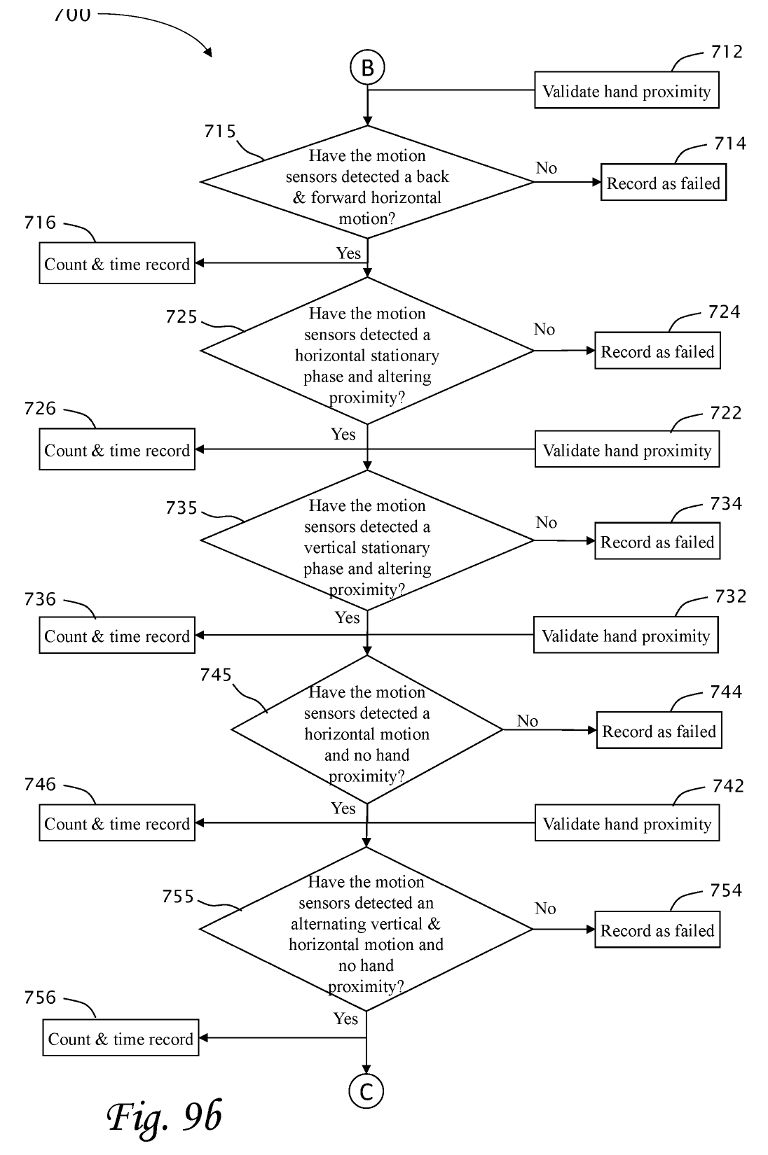

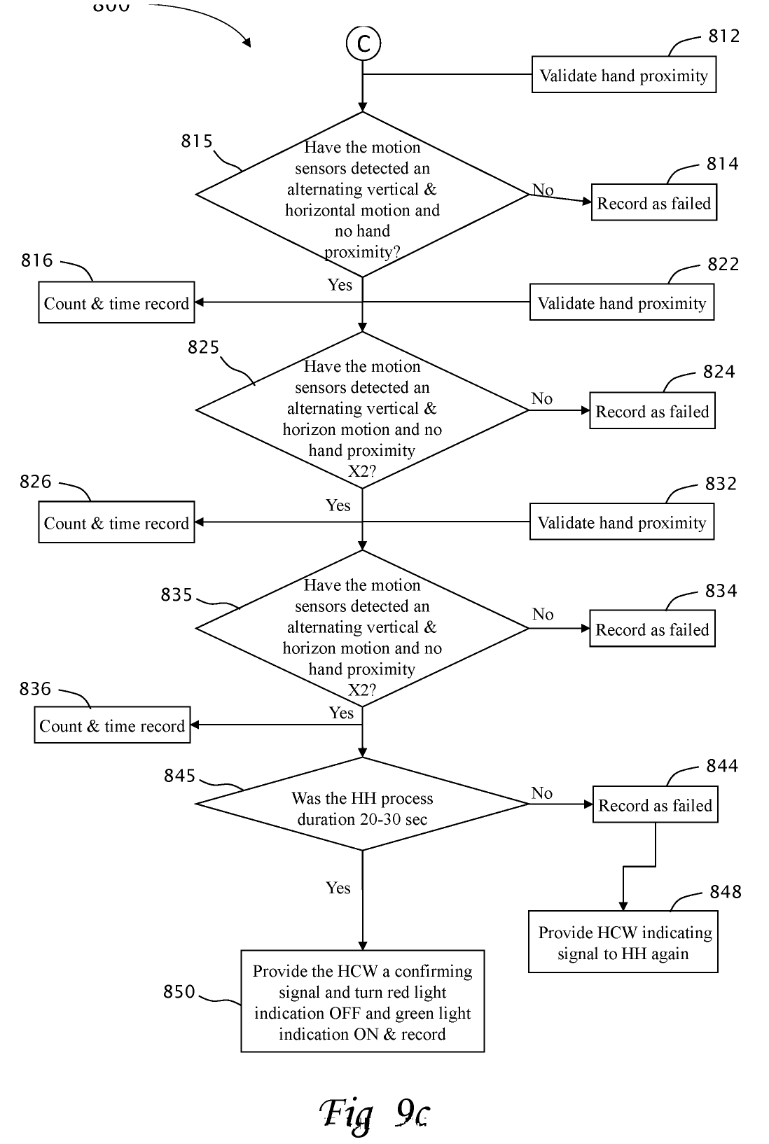

Reference is now also made to FIGS. 9a, 9b and 9c that combine to show a schematic flow chart of an exemplary HH-hands-motions-detection process 600, according to embodiments of the present invention. HH-hands-motions-detection process 600 determines if a HCW is performing the WHO hand hygiene procedures properly. It is made clear that the provided embodiments may include only parts of this scheme. Process 600 starts monitoring a HCW in step 601, when an action that brings about one of the WHO 5 Moments arises. Process 600 proceeds as follows:
Step 605: check if the HCW has used an HH solution.
   Infection-control-practices-monitoring system 100 determines if the HCW is near a solution bottle 120.
   In one embodiment of the present invention, each bottle 120 that contain the solution is labeled with a unique RFID tag 122. Thereby, when the HCW approaches bottle 120, RFID reader 240 of HCW PCMB 200 and/or RFID reader 330 of smart-wrist-wearable device 300 read RFID tag 122. Upon reading RFID tag 122, infection-control-practices-monitoring system 100 determines that the HCW is near the patient's HH solution bottle 120.
   The HCW positioning inside the health care facility may also use any indoor localization method known in the art including, with no limitations, systems based on GPS, GPRS, Wi-Fi, Wi-Fi positioning system (WPS) or WiPS/WFPS using GPS. Also cross-fit with the map of hospital beds and other positions of infection control practice.
   If infection-control-practices-monitoring system 100 determines that the HCW is not near HH solution bottle 120, keep trying the localization step for a preconfigured time interval (step 619).
Step 615: check if the HCW performed a Hand Hygiene.
   Infection-control-practices-monitoring system 100 determines if the HCW has already performed the HH in accordance to the WHO procedures. This is to avoid over washing of the hands, when not needed.
   To determine if the HCW has performed the HH in accordance to the WHO procedures, infection-control-practices-monitoring system 100 performs the steps outlined in FIG. 7.
   If infection-control-practices-monitoring system 100 determines if the HCW did performed the HH in accordance to the WHO procedures, go to step 699 (EXIT).
Step 625: check if the gyroscope indicates that a palm is facing up.
   Infection-control-practices-monitoring system 100 starts hand motions detection process.
   Infection-control-practices-monitoring system 100 determines if gyroscope 324 indicates that a palm is facing up. This can be determined if the monitored hand wearing smart-wrist-wearable device 300 is the hand destined to collect the dispensed solution. If the non-monitored hand is the hand destined to collect the dispensed solution, this can be detected using an imaging device 220 and verified by a temperature sensor that detects the cooling of the hand, after the solution comes in contact with the hand.
   If infection-control-practices-monitoring system 100 determines that a palm is facing up, go to step 645.
   If infection-control-practices-monitoring system 100 fails to detect that a palm is facing up, go to step 535.
Step 635: check a motion sensor indicated 1-3 vertical motions.
   Infection-control-practices-monitoring system 100 failed to detect that a palm is facing up.
   Therefore, infection-control-practices-monitoring system 100 determines if a motion sensor indicated 1-3 vertical motions. Infection-control-practices-monitoring system 100 interprets such detected motions as pressing on the bottle's head in order to dispense solution by the monitored hand wearing smart-wrist-wearable device 300.
   If infection-control-practices-monitoring system 100 fails to detect 1-3 vertical motions, the monitoring of hands motion may be terminated (in step 639).
Step 645: check if a sensor indicates that the hands of the HCW are in close proximity.
   Infection-control-practices-monitoring system 100 determined that solution bottle 120 was used by the HCW.
   It is assumed that while performing the WHO washing/rubbing routines. The wrists of both hands, must face each other at least part of the time, within a preconfigured time interval.
   Therefore, infection-control-practices-monitoring system 100 determines if a to sensor, typically a short range, living body proximity sensor 320, indicates that both hands are in close proximity.
   If infection-control-practices-monitoring system 100 fails to detect any that the hands of the HCW are in close proximity at least part of the time, the monitoring of hands motion may be terminated (in step 649).
Step 650: Activating HH monitoring.
   Once the pre requisites have been fulfilled, infection-control-practices-monitoring system 100 is set to start monitoring the sequential procedure of the WHO Handrub procedure 60, step by step. In each of the six active segments of the WHO Handrub procedure 60 (segments 2-7, segments 62-67 in FIG. 2b), the proximity between the two hands is continuously monitored and validated and the number of repeats in each of the nine segments and the time duration of each of the nine segments is recorded.
Step 655: check the proper performance of WHO Handrub segment 2 (62).
   Infection-control-practices-monitoring system 100 determines if the motion sensors detected a circular horizontal motion. The circular horizontal motions characterize WHO Handrub segment 2 (62).
   If infection-control-practices-monitoring system 100 has not detected circular horizontal motions or the two hands proximity validation (step 652) failed, record the failure (step 654) and go to step 699.
   Infection-control-practices-monitoring system 100 detected circular horizontal motions and the two hands proximity was validated.
   The number of circular horizontal motions and the time duration is recorded (step 656).
Step 665: check the proper performance of WHO Handrub segment 3 (63), first hand.
   Infection-control-practices-monitoring system 100 determines if the motion sensors detected back and forward horizontal motions, wherein a first hand is on top of the second hand. The back and forward horizontal motions characterize WHO Handrub segment 3 (63).
   If infection-control-practices-monitoring system 100 has not detected back and forward horizontal motions or the two hands proximity validation (step 662) failed, record the failure (step 664) and go to step 699.
   Infection-control-practices-monitoring system 100 detected back and forward horizontal motions and the two hands proximity was validated.
   The number of back and forward horizontal motions and the time duration is recorded (step 666).
Step 715: check the proper performance of WHO Handrub segment 3 (63), second hand.
   Infection-control-practices-monitoring system 100 determines if the motion sensors detected back and forward horizontal motions, wherein the second hand is on top of the first hand. The back and forward horizontal motions characterize WHO Handrub segment 3 (63).
   If infection-control-practices-monitoring system 100 has not detected back and forward horizontal motions or the two hands proximity validation (step 712) failed, record the failure (step 714) and go to step 699.
   Infection-control-practices-monitoring system 100 detected back and forward horizontal motions and the two hands proximity was validated.
   The number of back and forward horizontal motions and the time duration is recorded (step 716).
Step 725: check the proper performance of WHO Handrub segment 4 (64), vertical phase.
   Infection-control-practices-monitoring system 100 determines if the motion sensors detected vertical stationary phase motions and altering proximity, wherein a first hand is on top of the second hand. The vertical stationary phase motions characterize WHO Handrub segment 4 (64).
   If infection-control-practices-monitoring system 100 has not detected vertical stationary phase motions or the two hands altering proximity validation (step 722) failed, record the failure (step 724) and go to step 699.
   Infection-control-practices-monitoring system 100 detected vertical stationary phase motions or the two hands altering proximity validation.
   The number of vertical stationary phase motions and the time duration is recorded (step 726).
Step 735: check the proper performance of WHO Handrub segment 4 (64), horizontal phase.
   Infection-control-practices-monitoring system 100 determines if the motion sensors detected horizontal stationary phase motions and altering proximity, wherein a first hand is on top of the second hand. The horizontal stationary phase motions characterize WHO Handrub segment 4 (64).
   If infection-control-practices-monitoring system 100 has not detected horizontal stationary phase motions or the two hands altering proximity validation (step 732) failed, record the failure (step 734) and go to step 699.
   Infection-control-practices-monitoring system 100 detected horizontal stationary phase motions or the two hands altering proximity validation.
   The number of horizontal stationary phase motions and the time duration is recorded (step 736).
Step 745: check the proper performance of WHO Handrub segment 5 (65), first thumb.
   Infection-control-practices-monitoring system 100 determines if the motion sensors detected a horizontal motion and no hand proximity, wherein the second hand fingers are hooked with the first hand fingers. The horizontal motion and no hand proximity characterize WHO Handrub segment 5 (65).
   If infection-control-practices-monitoring system 100 has not detected horizontal motions and no hands proximity validation (step 742) failed, record the failure (step 744) and go to step 699.
   Infection-control-practices-monitoring system 100 detected horizontal motions and no hands proximity was validated.
   The number of horizontal motions and the time duration is recorded (step 746).
Step 755: check the proper performance of WHO Handrub segment 5 (66), first thumb.
   Infection-control-practices-monitoring system 100 determines if the motion sensors detected alternating vertical and horizontal motions and no hand proximity, wherein the second hand fingers wrap the first hand thumb. The alternating vertical and horizontal motion and no hand proximity characterize WHO Handrub segment 6 (66).
   If infection-control-practices-monitoring system 100 has not detected back and forward horizontal motions and no hands proximity validation (step 752) failed, record the failure (step 754) and go to step 699.
   Infection-control-practices-monitoring system 100 detected the alternating vertical and horizontal motion and no hands proximity was validated.
   The number of The alternating vertical and horizontal motions and the time duration is recorded (step 756).
Step 815: check the proper performance of WHO Handrub segment 6 (66), second thumb.
   Infection-control-practices-monitoring system 100 determines if the motion sensors detected alternating vertical and horizontal motions and no hand proximity, wherein the first hand fingers wrap the second hand thumb. The alternating vertical and horizontal motion and no hand proximity characterize WHO Handrub segment 6 (66).
   If infection-control-practices-monitoring system 100 has not detected back and forward horizontal motions and no hands proximity validation (step 812) failed, record the failure (step 814) and go to step 699.
   Infection-control-practices-monitoring system 100 detected the alternating vertical and horizontal motion and no hands proximity was validated.
   The number of The alternating vertical and horizontal motions and the time duration is recorded (step 816).
Step 825: check the proper performance of WHO Handrub segment 7 (67), first hand.
   Infection-control-practices-monitoring system 100 determines if the motion sensors detected alternating vertical and horizontal motions and no hand proximity, wherein the second hand fingers wrap the first hand thumb. The alternating vertical and horizontal motion and no hand proximity characterize WHO Handrub segment 7 (67).
   If infection-control-practices-monitoring system 100 has not detected back and forward horizontal motions and no hand proximity validation (step 822) failed, record the failure (step 824) and go to step 699.
   Infection-control-practices-monitoring system 100 detected back and forward horizontal motions and no hand proximity was validated.
   The number of back and forward horizontal motions and the time duration is recorded (step 826).
Step 835: check the proper performance of WHO Handrub segment 7 (67), second hand.
   Infection-control-practices-monitoring system 100 determines if the motion sensors detected alternating vertical and horizontal motions and no hand proximity, wherein the first hand fingers wrap the second hand thumb. The back and forward horizontal motions characterize WHO Handrub segment 7 (67).
   If infection-control-practices-monitoring system 100 has not detected back and forward horizontal motions and no hand proximity validation (step 832) failed, record the failure (step 834) and go to step 699.
   Infection-control-practices-monitoring system 100 detected back and forward horizontal motions and no hand proximity was validated.
   The number of back and forward horizontal motions and the time duration is recorded (step 836).
Step 845: check if the HH process total duration was 20-30 sec.
   Infection-control-practices-monitoring system 100 determines if the HH process total duration was 20-30 sec.
   If infection-control-practices-monitoring system 100 has determined that the HEI process total duration was not 20-30 sec, record the failure (step 844) and provide the HCW indicating signal to perform the HH again (step 848).
Step 850: Provide a success indication and report.
   Infection-control-practices-monitoring system 100 has determined that the WHO HEI procedure has been performed successfully. Therefore, infection-control-practices-monitoring system 100 issues a success indication to the HCW, for example and audible of vibratory signal. Furthermore, infection-control-practices-monitoring system 100 turns ON green light on 260g the PCMB device 200 to warn the HCW and patient, and updates data logging module 280.
Step 699: Exit.
[end of process 600]

Processes 400, 500 and 600 are further summarized in the following table 2 and group 2600 of iconic drawings in FIG. 22:

TABLE 2

| | | | |
|---|---|---|---|
| Referring to HH | WHO Handrub step | WHO Handrub step 3 | WHO Handrub |
| solution bottle | 2 (62), iconic | (63), iconic drawing | step 4 (64), |
| 120 and AHRB | drawing 2610. | 2620. | iconic drawing |
| usage sensor | 1 or 2 sensors | One or both sensors | 2630. |
| 1520. | moving in a | are facing upwards, | The used |
| Detect a sharp | horizontal or at a | one or both sensors are | sensor/s are in a |
| up and down | small angle from the | horizontal or at a small | vertical position |
| motion of one of | horizon in a circular | angle from the horizon. | and are moving |
| the sensors, the | or elliptic motion. | 2 sets of 2-3 stroke | forward and |
| sensor is in a | In case of 2 sensors | horizontal motions are | backwards |
| horizontal | the motion of | detected. In case of 2 | strokes or in an |
| position, | sensors is in | sensors, the length of | upwards and |
| Validate it with | opposite directions. | the strokes is almost | downwards |
| the pressure | | the same, in case of 1 | strokes. |
| sensor on the AHRB | | sensor the lengths of | |
| pump. | | the strokes are | |
| | | significantly different. | |
| | WHO Handrub step | WHO Handrub step 6 | WHO Handrub |
| | 7 (67), iconic | (66), iconic drawing | step 5 (65), |
| | drawing 2660. | 2650. | iconic drawing |
| | When 1 sensor is | When 1 sensor is being | 2640. |
| | being used then the | used then the sensor is | The sensor/s are |
| | sensor is at an | at an almost horizontal | in a semi |
| | almost vertical | position facing | horizontal |
| | position, several | upwards then | position facing |
| | rapid vertical | according to the hand | up wards or 1 |
| | rotating motions are | the sensor is wore on | upwards and 1 |
| | detected and after a | 2-3 strokes are | downwards |
| | short pause a second | performed: | position, after 2-3 |
| | set of such motion | Senor on left hand: an | strokes the |
| | is detected the 2 sets | initial downward | sensor changes |
| | of motions have a | motion is detected | the sensor/s |
| | different vertical | followed by 2-3 | changes the |
| | radius of motion. | upwards rotating | plain of their |
| | When 2 sensors are | strokes are detected | facing upwards |
| | used, both of them | after a short pause a | changes to |
| | are in an almost | second set of similar | downwards and |
| | vertical position one | motions are detected. | vice versa and a |
| | of the sensors starts | Sensor on right hand: a | 2^{nd} set of 2-3 |
| | a rotating vertical | set of 2-3 downward | strokes are |
| | motion and in a | rotating strokes are | performed. |
| | short time after the | detected and after a | |
| | second sensor starts | short pause a set of 2-3 | |
| | a vertical rotating | upwards strokes are | |
| | motion and both are | detected. | |
| | moving in the same | When 2 sensors are | |
| | speed. After a short | used a set of 2-3 | |
| | pause the sensors | rotating strokes in | |
| | motion changes and | opposite directions | |
| | the initiating sensor | (upwards/downwards) | |
| | changes between | are detected and after a | |
| | the sensors. | small pause a 2^{nd} set of | |
| | | rotating strokes in | |
| | | opposite directions are | |
| | | detected. | |

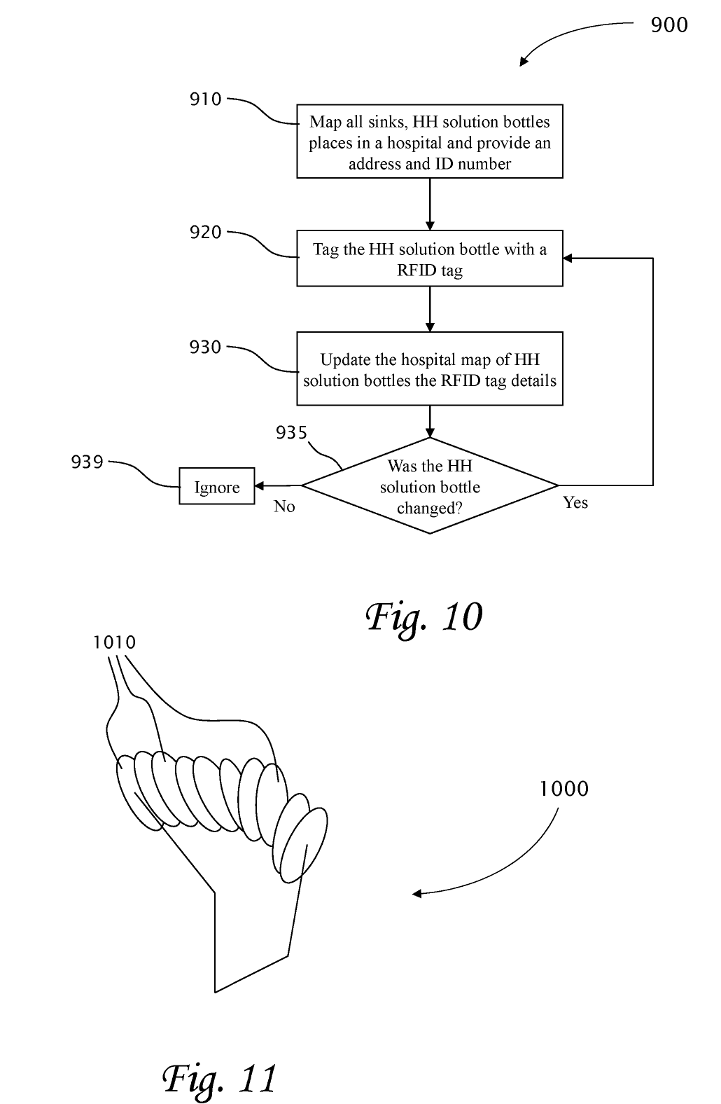

Reference is now also made to FIG. 10 that outlines a schematic flow chart of an exemplary RFID-tagging process 900 for tagging HH solution bottle and patient, according to embodiments of the present invention. Process 900 proceeds as follows:
Step 910: mapping.
   Map all sinks, HH solution bottles places in a hospital and provide an address and ID number for each element.
Step 920: Tag the HH solution bottle with a RFID tag.
   Tag the HH solution bottle with a unique RFID tag.
Step 930: Update the hospital map.
   Update the hospital map with the HH solution bottles the RFID tag details.
Step 935: check if a HH solution bottle was changed.
   Check if a HH solution bottle was changed.
   If a HH solution bottle was changed, go to Step 920.
[end of process 900]
Hand Hygiene Training System

It is an aspect of the present invention to provide system, devices and methods for training and HCW to perform proper Hand Hygiene.

The described hand motion detection and interpretation algorithm described in by processes 400, 500 and 600, as well as in Table 2, can be used along with detection of additional motions for the training of an antiseptic hand washing training program.

One of the aspects of the present invention is about an automated, real time self-training system and method that enables a HCW to perform hand hygiene training and auditing in accordance to the 5 Moments recommended by the WHO and the set of hand motions required by the WHO to complete a properly performed hand hygiene that covers all hand surfaces.

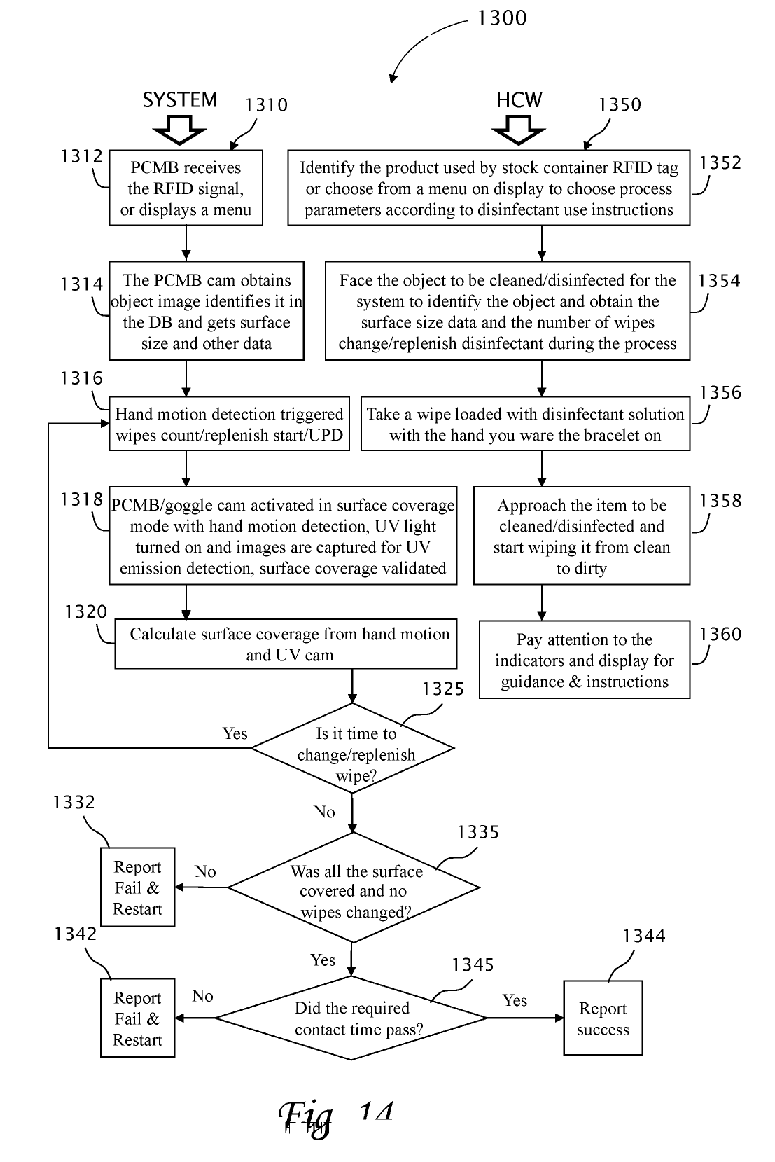

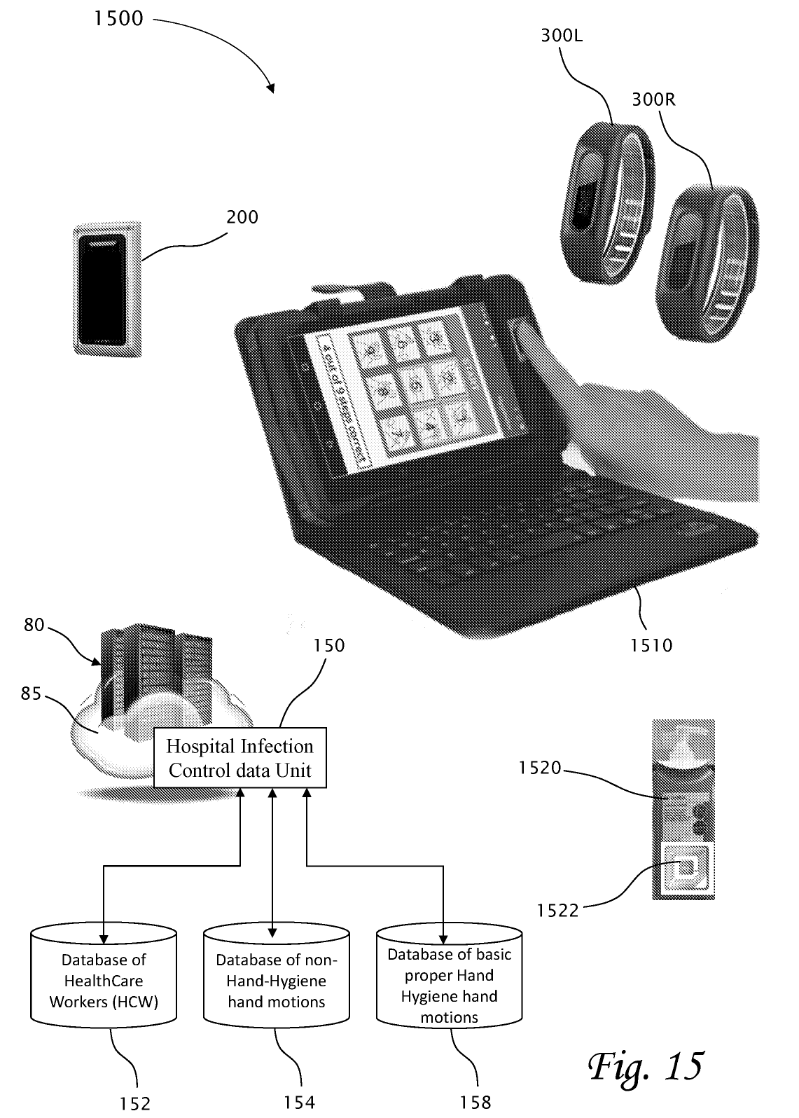

Reference is made back to the drawings. FIG. 15 is a schematic system diagram illustration of a basic infection-control-practices-training system (ICPTS) 1500, according to the embodiments of the present invention. In the example shown in FIG. 15, infection-control-practices-training system 1500 includes a PCMB device 200, introduced before, that is associated with a particular HCW and is coupled to operate with that HCW, a first smart-wrist-wearable device 300R and preferably a second smart-wrist-wearable device 300L (for the second hand), a computerized device 1510 such as, with no limitation, a laptop or a tablet, a first local communication network 85 facilitating computerized device 1510 to communicate with an infection control unit 150 of the health facility servers 80, a second local communication network, such as Bluetooth (BT) facilitating computerized device 1510 to communicate with smart-wrist-wearable device 300 and preferably, at least one hand hygiene (HH) solution bottle 120 having a RFID tag 122 associated therewith. Infection control unit 150 further includes a health-care-workers database 152, a non-Hand-Hygiene hand motions database 154, a Hand-Hygiene hand motions database 158 and optionally, additional databases. Smart-wrist-wearable device 300 may include time measuring,

Infection-control-practices-training system 1500 provides the training HCW real time indication and corrective guidance to the hand motions that were performed in a wrong manner or were missed, and that enables the HCW to fix mistakes during the training session. The system can audit the training of the HCW in real time and only after the HCW completed all required steps in a correct manner several times in a raw he/she will be provided a certification number. Infection-control-practices-training system 1500 enables the facility to require from the HCW stuff periodic training and auditing sessions thus improving the quality of the performed hand hygiene within the healthcare facility and reduce the nosocomial infections rate in the facility. The clear advantages of infection-control-practices-training system 1500 and its impact on hand hygiene quality of performance include but is not limited to:
Ensures a standard and uniform training performance of hand hygiene according to a validated procedure.
Enables periodical training and auditing of HCW.
Enables self-training of HCW day and night in between tasks.
Ensures that all HCW are trained at all times.
Eliminates the need for hand hygiene training and auditing by human resources.
And more.

Once collected, the motion data and other data collected by the sensing devices is analyzed by a special algorithm in view of the motions described in WHO charts. The total procedure time is measured as well and the duration and number of motions during the performance of a certain WHO recommended motion or the entire process that has to be done within 15-30 seconds.

When a certain HCW performs an initial training using infection-control-practices-training system 1500, the algorithm analyzes the process at the beginning with a set of basic structured outlined motions with a certain range of freedom as the user starts to train infection-control-practices-training system 1500 begins a machine learning process that learns the particular HCW's variations of hand motions and narrows down the initial degrees of freedom till it becomes able to identify that HCW according to his/her specific hand hygiene motions or combination thereof. The range of variations that are allowed may include the angle of the hands related to the earth, speed of performance length of stokes, direction of strokes etc., that constitute the personal interpretation and behavior of that HCW but not the basic WHO required gross motions and their described way of performance and the motions order. Regarding the possible algorithms, they may include an analysis of the hand motion in space relative to fixed forces such as earth gravity and the relative position of the gyroscope, and may combine the data from the accelerometer and other sensors and their relative position. The hand motion detection and interpretation algorithm described in table 2, with respect to infection-control-practices-monitoring system 100, with the addition of other several motions can be used for the training of Antiseptic Hand Washing training.

The training process itself is designed for 3 levels of training, Novice, Advanced and Audit. After identifying the user, each of the levels provides accesses to different training modules that include the required training steps. In case an HCW fail to pass, in the chosen module, the Audit, the trainee is referred to a lower level of training to remind and train him again.

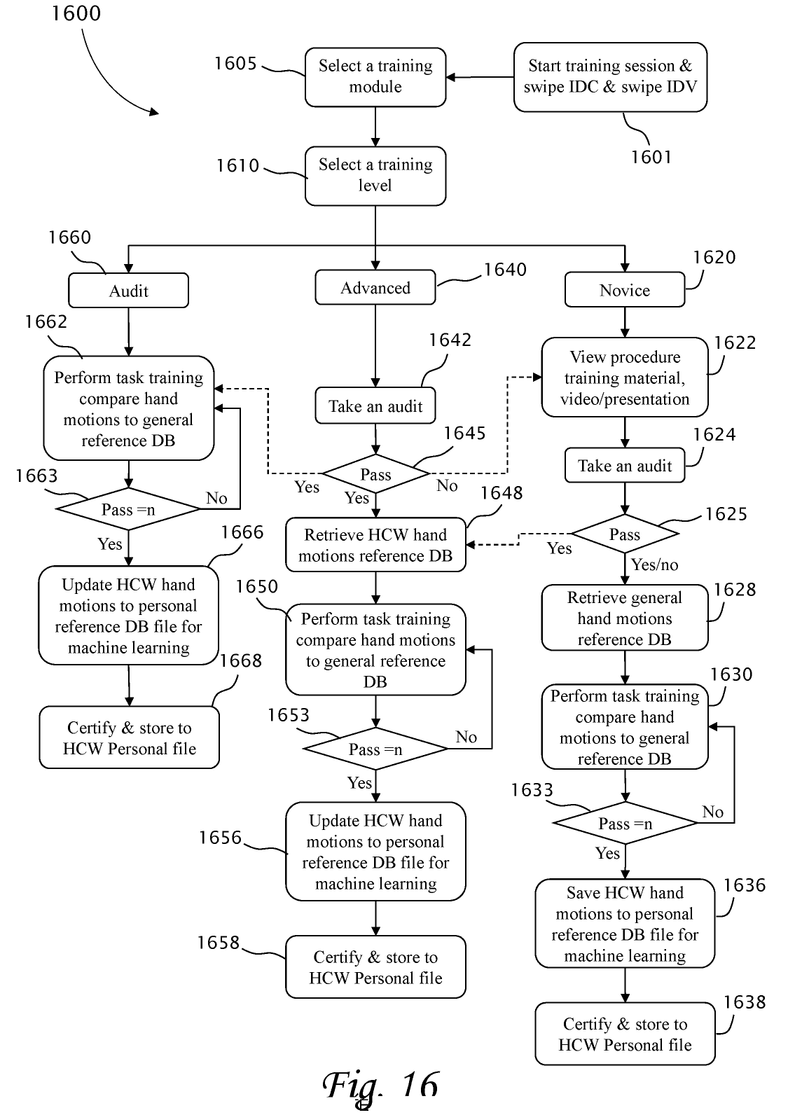

Reference is now also made to FIG. 16, showing a schematic flow chart of an exemplary cleaning-and-disinfecting process 1600 for determining that a HCW performed the cleaning-and-disinfecting process properly, according to embodiments of the present invention. It is made clear that the provided embodiments may include only parts of this scheme. Process 1600 starts a training session when a HCW activates the system by pressing a start button and/or swiping an identification card (IDC) and/or swiping an IDC in step 1601. Process 1600 proceeds as follows:
Step 1605: Select a training module.
   The HCW selects a training module: Hand hygiene training, gloving training, isolation training or environment care.
Step 1610: Select a training level.
   The HCW Select a training level: Novice, Advanced or Audit.
Step 1620: Novice level.
Step 1622: View procedure training material, video/presentation.
   The HCW learns and/or reviews the theory of the selected module.
Step 1624: take an audit.
   The HCW takes an audit to find out his/her performance level.
Step 1625: check if passed.
   Infection-control-practices-training system 1500 determines if the HCW passed the audit. If passed, the HCW may proceed with step 1648 of the Advanced level.
Step 1628: Retrieve general hand motions reference DB.
   Infection-control-practices-training system 1500 retrieves the general hand motions reference DB containing the hand motions data related to the selected module.
Step 1630: Perform task training compare hand motions to general reference DB.
   The HCW performs task training session, wherein infection-control-practices-training system 1500 compares hand motions to general reference DB to thereby determines if the motions were performed in order or not.
Step 1633: check if passed.
   Infection-control-practices-training system 1500 determines if the HCW performed the hand motion task correctly. If not passed, the HCW may try again (go to step 1630) n more times, for example 2 more times.
Step 1636: save the HCW hand motions data.
   Infection-control-practices-training system 1500 has determined that the HCW performed the hand motion task correctly.
   Infection-control-practices-training system 1500 saves the HCW hand motions data in the personal reference DB file of that HCW, for machine learning purposes.
Step 1638: Certify & store to HCW Personal file.
   Infection-control-practices-training system 1500 certifies and stores the HCW hand motions related data to in the HCW Personal file.
Step 1640: Advanced level.
Step 1642: take an audit.
   The HCW takes an audit to find out his/her performance level.
Step 1645: check if passed.
   Infection-control-practices-training system 1500 determines if the HCW passed the audit.
   If passed, the HCW may proceed with step 1662 of the Audit level.
   If failed, the HCW may proceed with step 1622 of the Novice level.
Step 1648: Retrieve the HCW hand motions reference DB.
   Infection-control-practices-training system 1500 retrieves the HCW hand motions reference DB containing the hand motions stored data related to the selected module.
Step 1650: Perform task training compare hand motions to general reference DB.
   The HCW performs task training session, wherein infection-control-practices-training system 1500 compares hand motions to general reference DB to thereby determines if the motions were performed in order or not.
Step 1653: check if passed.
   Infection-control-practices-training system 1500 determines if the HCW performed the hand motion task correctly. If not passed, the HCW may try again (go to step 1650) n more times, for example 2 more times.
Step 1656: update the HCW hand motions data.
   Infection-control-practices-training system 1500 has determined that the HCW performed the hand motion task correctly.
   Infection-control-practices-training system 1500 updates the HCW hand motions data in the personal reference DB file of that HCW, for machine learning purposes.
Step 1658: Certify & store to HCW Personal file.
   Infection-control-practices-training system 1500 certifies and stores the HCW hand motions related data to in the HCW Personal file.
Step 1660: Audit level.
Step 1662: Perform task training compare hand motions to general reference DB.
   The HCW performs task training session, wherein infection-control-practices-training system 1500 compares hand motions to general reference DB to thereby determines if the motions were performed in order or not.
Step 1663: check if passed.
   Infection-control-practices-training system 1500 determines if the HCW performed the hand motion task correctly. If not passed, the HCW may try again (go to step 1650) n more times, for example 2 more times.
Step 1666: update the HCW hand motions data.
   Infection-control-practices-training system 1500 has determined that the HCW performed the hand motion task correctly.
   Infection-control-practices-training system 1500 updates the HCW hand motions data in the personal reference DB file of that HCW, for machine learning purposes.
Step 1668: Certify & store to HCW Personal file.
   Infection-control-practices-training system 1500 certifies and stores the HCW hand motions related data to in the HCW Personal file.
[end of process 1600]

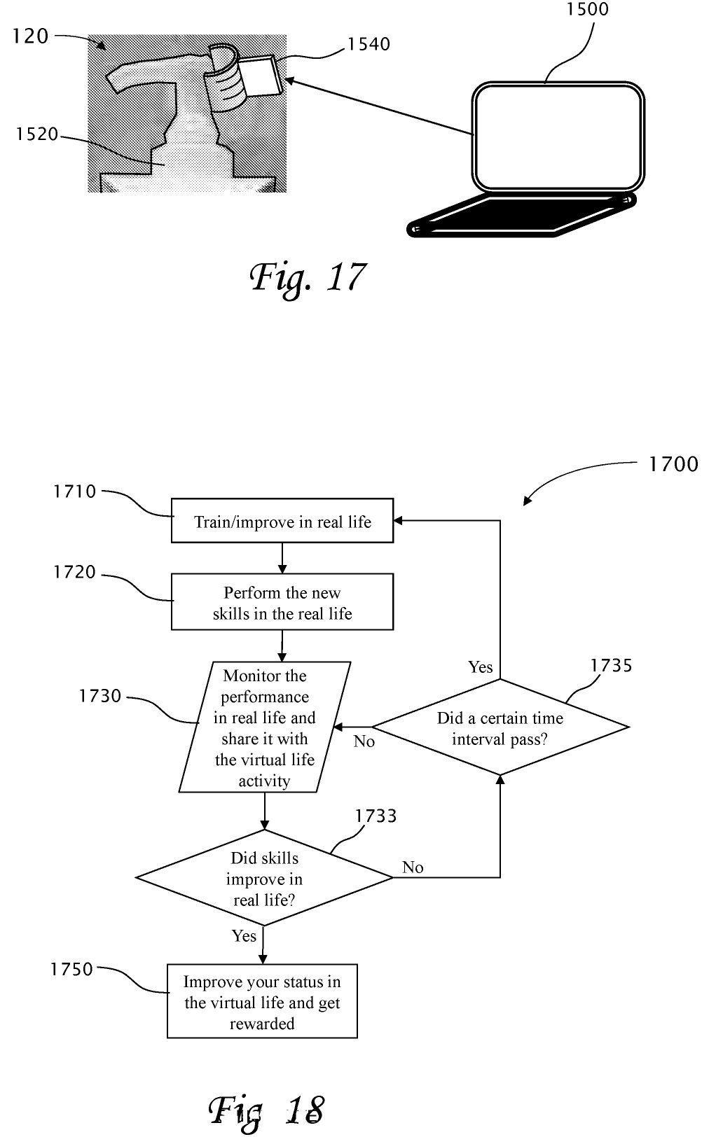

It should be noted that additional possible sensors connected to infection-control-practices-training system 1500, may be used. The additional sensors may include a pressure sensing device (not shown), being a AHRB usage sensor, that can be embodied as a plate or patch attached to the bottom of HH solution bottle 120. In another embodiment, the AHRB usage sensor 1520 can be in the form of a horse shoe shaped spring 1540 (see FIG. 17) that closes an electrical circuit when pressed fully down as can be seen from the following drawing. AHRB usage sensor 1520 can be made of silicone or other flexible polymer reinforced with metal wire inside and placed on the neck of the AHRB/antiseptic pump and it detects when the pump is pressed if the right volume of AHRB was dispensed for the hand hygiene process. The AHRB usage sensor can be connected to the training system by wire or wireless.

When the HCW uses bottle 120, it is done by predictable hand motions. The HCW PCMB 200, using the sensors and the sensors, such as accelerometer 322, detects the performed non-Hand-Hygiene hand motion and matches the detected non-Hand-Hygiene hand motion is matched against a database of non-Hand-Hygiene hand motions. Thereby, infection-control-practices-monitoring system 100 determines if the HCW has used an HH solution.

It should be noted that infection-control-practices-training system 1500 has the ability to further increase the type of modules for other areas of training within the infection control.

Infection-control-practices-training system 1500 further includes an application that enables a user to train with the process of PPE wearing in a mock process initiated by isolation cards with a RFID tag on it and a set of boxes labeled with RFID tags with the different PPE items. The HCW training with the system obtains the approval of passing the training after performing correctly 3 procedures in a row.

The collected hand motions the training-HCW performed during the training session will be used by the system for machine learning in order to create a specific user set of hand motions for a more accurate hand motions detection for the monitoring system.

An “Inversed Simulator” Real Life Social Game Application to Improve skills.

Another aspect of the present invention is a method and system for hand hygiene HCW performance improvement and increase hand hygiene compliance through an “ inversed simulator” real life integrated dedicated social game application to improve hand hygiene skills.

All these means that are used to promote the hand hygiene in hospitals tend to create objections among the HCW to the training requirement and the monitoring. To overcome these objections and assist with the introduction and the implementation of an electronic hand hygiene training system and a hand hygiene electronic system which was described elsewhere, the turn the entire process into fun and competitive social game among the users and the health facilities, is suggested.

One of the aspects of the present invention is about creating a system that enables to train in real life in order to improve the results in the virtual life based on the results in real life the present invention is about an “inversed simulator”. In a simulator one trains in a virtual environment to improve himself/herself in real life. The invention here is about training and practicing in real life to improve himself/herself in and through the virtual life in order to maintain the acquired skills in real life again. The existing simulator training technique is based on the user training in a simulated virtual environment with performance in this environment data collection and audit. When a person training on a simulator after he acquires a certain level of performance he is certified and allowed to move to real life performance of those skills.

The present invention is based on an initial training of hand hygiene performance and quality according to the WHO charts in real life that is then continuously monitored by an integrated real-time monitoring system that monitors the performance of hand hygiene and its quality (described elsewhere). The data generated from the hand hygiene monitoring is then retrieved by the gaming application and the improvement in real life is translated into improvement in the virtual life of the game if the HCW game results are not adequate for a certain period of time the HCW is advised to return to the real life and re-train in order to improve his hand hygiene capabilities.

The present invention is based on an initial training of hand hygiene performance and quality, according to the WHO charts in real life that is then continuously monitored by an integrated real-time monitoring system that monitors the performance of hand hygiene and the quality thereof, as described herein. The data generated from the hand hygiene monitoring is then retrieved by the gaming application and the improvement in real life is translated into improvement in the virtual life of the game. If the HCW game results are not adequate for a certain period of time, the HCW is advised to return to the real life and retrain, in order to improve his hand hygiene capabilities.

Reference is now also made to FIG. 18, showing a schematic flow chart of an exemplary an inversed-simulator process 1700 for transferring performance and quality achievements of a HCW form “real life” to “virtual life”, according to embodiments of the present invention. Process 1700 proceeds as follows:
Step 1710: Train/improve in real life.
   The HCW continues his real life activity, using infection-control-practices-training system 1500.
Step 1720: Perform the new skills in the real life.
   The HCW performs new skills, using infection-control-practices-training system 1500, to thereby attempting to improve his skill.
Step 1730: Monitor the performance in real life and share it with the virtual life activity.
   Infection-control-practices-training system 1500 monitors and evaluate the activity of the HCW.
Step 1733: check if the skills improved in real life.
   Infection-control-practices-training system 1500 determines if the HCW performance of the HCW has improved in real life.
   If infection-control-practices-training system 1500 has determined that the performance of the HCW has improved in real life, go to step 1750.
Step 1735: check if a certain time interval has passed.
   Infection-control-practices-training system 1500 has determined that the performance of the HCW has not improved in real life.
   Infection-control-practices-training system 1500 determines if a certain time interval has passed.
   If the passed time interval is greater that a preset threshold, go to step 1710.
   Go to step 1730.
Step 1750: Improve the status in the virtual life and get rewarded.
   Upon improvement in performance/quality, the HCW is rewarded.
[end of process 1700]

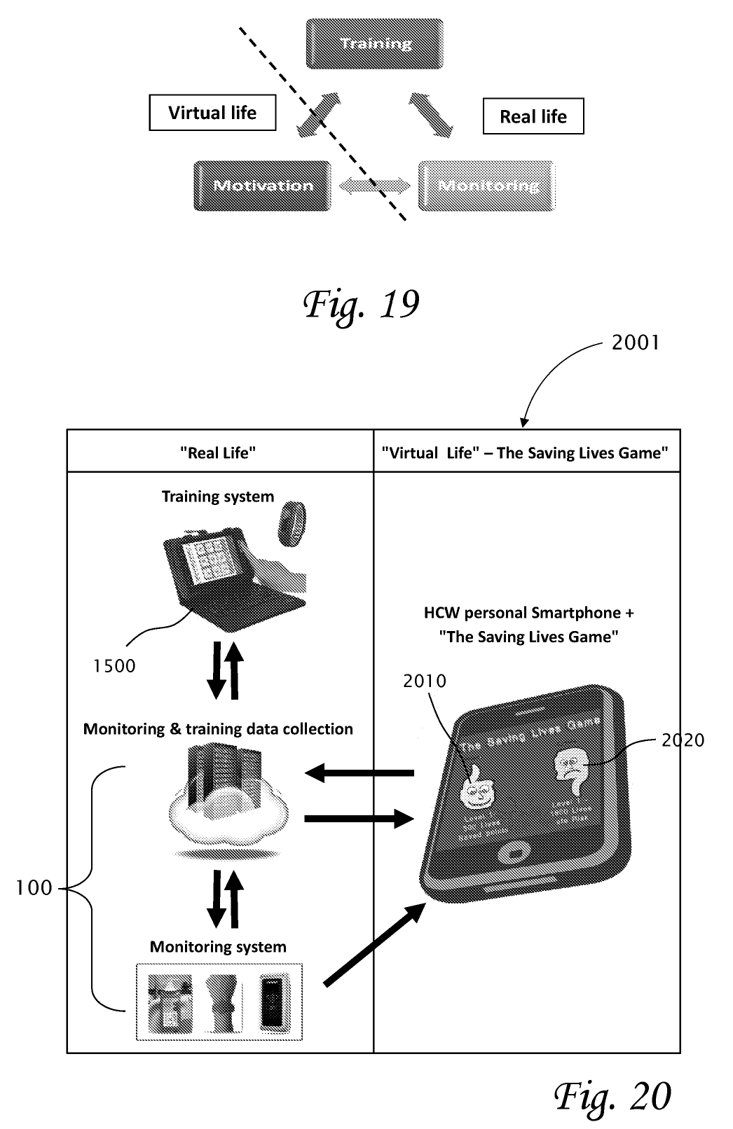

One of the aspects of the present invention is the “Saving Lives” mobile application social game 2000 (see FIG. 20), for enhancing hand hygiene compliance and motivation, among health care workers. FIG. 19 is a schematic system diagram illustration the motivation relationship between the HCWs and the Infection-control-practices-training/monitoring systems.

The purpose of the social game is to promote correct hand hygiene (HH) compliance and hand hygiene performance, among health care workers (HCW) through a competitive social game. The game application is installed on the personal smartphone/tablet of each and every player. The game is played in combination with “The Monitor”—HH quality real time Infection-control-practices-monitoring system 100 and “The Trainer”—a hand hygiene real time, self-coaching infection-control-practices-training system 1500.

The concept behind Saving Lives Game 2000 is based on the assumption that a social game played among HCW in a competitive manner can lead to improved personal HH compliance and to improvement of HH compliance on the department level, on the hospital level and so on, thus turning the “annoying” HH into a habit through a fun game and reducing objections among HCW to the new technologies of electronic training and electronic monitoring.

The game 2000 structure and design is to encourage the personal and group performance of:
A correct hand hygiene (according to the WHO 9 Steps).
At the right time (according to the WHO 5 Moments)
At all times.
By all HCW.
From one ward to the whole world

These goals are achieved through the competitive social game 2000 that is based on collecting a “Life Saved” symbol 2720 (shown in FIG. 23) or every time a correctly performed HH at the right moment was done by the HCW, or, on the other hand, getting a “Life at Risk” symbol 2710 (shown in FIG. 23) every time the HCW missed a HH or performed the HH not according to the 9 steps. The info regarding the quality of HH performance is obtained from the hand hygiene monitoring system that monitors the HCW HH during his work.

Game 2000 is played on a personal level and on a ward level at the same time as HH on a ward is a team work and depends on leadership and team work. Based on this assumption the personal scores and achievements can influence and be influenced by the person's actions and to a certain extent by the ward personnel from the head of the department to last new on board nurse.

The participants of game 2000 have a personal score accumulation and a departmental score accumulation the participant first goal is the collect as many as possible points in order to advance themselves to a higher level. With each personal level accomplished by the HCW a prize is won, such as, with no limitation, a medal, and an appreciation card or letter and the HCW name and picture is published within the winner's board (in the hospital and on the application), a department trophy etc. and so on up to the big prize that is on the global level with media and PR.

Game Installation and Connection:

Each participant should download the application from the Appstore or Googleplay and install it on his/her smart phone (SF), create an avatar with a user name and create a connection to the personal communication unit of infection-control-practices-monitoring system 100 to obtain real-time hand hygiene data collected. The application connects to the cloud servers 80 of the monitoring and gaming system as well. The monitoring data is made available immediately to the personal game but for the rest of data and comparison the data on cloud servers 80 are used.

The Rules of the Game:

Anny participant connected to the game and to The personal monitoring system becomes part of the HH monitoring and real time training in the “real world” and a competitor in the “virtual world”.

The participant performance of HH is being constantly monitored as the participant joins the game the he enters at Level 1 and then moves to higher levels when certain conditions are fulfilled.

Level 1 Rules and Requirements:

1. Each time the participant HCW performs a correct HH at the right moment a signal is sent from The personal monitoring system to the participant smart phone and “Life Saved” symbol appears on the participant SF display. Only if the participant has performed 2 successive successful HH procedures and has received 2 “Life Saved” in a row he gets a point that accumulates as the pairs of successful HH events accumulates. If the HH procedure was poorly performed or was not performed at all the participant receives a “Life at Risk” symbol and gets a negative point that accumulates as these events accumulate. In order for the participant to move to the next level he/she needs to reach a total of, for example, 1000 points.

Level 2 Rules and Requirements:

2. The participant gets, for example, 1 point for every, for example, 4 successive successful HH procedures performed. In order for the participant to move to the next level he needs to accumulate, for example, 100 points. For the failed HH the participant gets, for example, 1 point.

Level 3 Rules and Requirements:

4. The participant gets, for example, 1 point for every, for example, 16 successive successful HH procedures performed. In order for the participant to move to the next level he needs to accumulate, for example, 10 points. For the failed HH the participant gets, for example, 1 point.

Level 4 Rules and Requirements:

4. The participant gets, for example, 1 point for every, for example, 256 successive successful HH procedures performed. In order for the participant to move to the next level he needs to accumulate 1 point. For the failed HH the participant gets 1 point.

The Competition Rules and Requirements:

All participants of the participants of the “Life Saving game” are participating at the same time in competitions on several levels—personal to international.

Personal competition—the participant competes on the local level with his colleagues to the department

Hospital level competition—the hospital wards score of their participants are compared and 3 winning places are rewarded. To determine the wining ward, the “Lives at Risk” points are taken in account and they influence the total score for wining. The wining departments get 3 different levels of prizes. For example: 1st place—a week end at a spa resort, 2nd place a day trip to a special place, 3rd a dinner party at a special restaurant.

National level competition—all hospitals participating in the game are eligible to participate in the national level competition with the 3 wining departments of each hospital. The national level competition is a competition for “the next year” that means that each hospital's 3 this year wining wards are participating in the successive year competition and based on their next year results the 3 wining hospitals are determined and they win a prize with PR and national media coverage.

International level competition—all last year wining 3 hospitals on national level can participate on the International level competition that will take place on the successive year the participating hospitals will have to maintain or improve their last year results in order to win this competition. The 3 wining hospitals will be announced in the international media and will be awarded a special certificate and a trophy by the WHO and will get international media coverage.

Every participant that passed level 4 becomes “Master of Saving Lives” and gets a special trophy, a certificate and a special pin and becomes eligible to participate in the “International Masters” continuation part of the game in this part of the game participants are encouraged to maintain their achievements and improve them by collecting stars thus becoming “One Star Master of Saving Lives” in order to get the stars the following points should be achieved:

For 1 star—a total of 512 successive HH procedures has to be performed. With every missed or poorly performed the counting stars from scratch.

For 2 star—a total of 1024 successive HH procedures has to be performed. With every missed or poorly performed the counting stars from scratch.

For 3 star—a total of 2048 successive HH procedures has to be performed. With every missed or poorly performed the counting stars from scratch.

For 4 star—a total of 4096 successive HH procedures has to be performed. With every missed or poorly performed the counting stars from scratch.

For 5 star—a total of 8192 successive HH procedures has to be performed. With every missed or poorly performed the counting stars from scratch.

Those that have reached the stars' level of game will be decorated and their info published within the game participants and they will be decorated as “Life Saving Champions” every year they will be invited to an annual meeting during which the new Champions are announced and decorated each year the 3 champions that has the highest scores are selected and decorated as Champions of the Champions and 3 prizes are given to them with international media coverage. The entire event will take place under the sponsorship of the WHO.

Components and data flow between the “Real Life” and the “Virtual Life” for hand hygiene training, monitoring, and motivating system is shown in FIGS. 19 and 20.

The present invention being thus described in terms of several embodiments and examples, it will be appreciated that the same may be varied in many ways. Such variations are not to be regarded as a departure from the spirit and scope of the invention, and all such modifications as would be obvious to one skilled in the art are considered.

1. An infection-control-practices-monitoring system for monitoring hand motions of a health care worker (HCW), the hand motions are related to infection control practices, the system comprising:
a) at least one smart-wrist-wearable device adapted to be worn on a human wrist of a first hand;
b) a bracelet-communication configured to communicate with any device sharing same the communication protocols; and
c) a data repository unit,
wherein said at least one smart-wrist-wearable device includes a living body proximity sensor, an accelerometer, a gyroscope and a communication unit for communicating with said data repository unit;
wherein said living body proximity sensor is configured to detect a live tissue at a short range of up to 50 centimeters;
wherein at least one of said accelerometer, said gyroscope and said living body proximity sensor detects hand motions of the health care worker; and
wherein said smart-wrist-wearable device is configured to perform a hand motion analysis to thereby analyze the detected hand motions of the health care worker, and to thereby determine if said hand motion were performed in compliance with a preset required hand motion sequence and rate.

2. The infection-control-practices-monitoring system of claim 1, wherein said data repository unit includes a database of known hand motions, and wherein said hand motion analysis includes matching said detect hand motions with said known hand motions.

3. (canceled)

4. The infection-control-practices-monitoring system of claim 1, wherein said at least one smart-wrist-wearable device further includes a RFID reader.

5. The infection-control-practices-monitoring system of claim 1, wherein said at least one smart-wrist-wearable device further includes an indicator.

6. (canceled)

7. The infection-control-practices-monitoring system of claim 1, wherein said living body proximity sensor is selected from group including a thermal sensor, an RLC sensor, an ultrasound sensor and a combination thereof

8. The infection-control-practices-monitoring system of claim 1, wherein said living body proximity sensor is capable of sensing the second hand of the HCW.

9. The infection-control-practices-monitoring system of claim 1 further comprising a personal-communication-monitoring-badge device comprising:
a) a processing unit;
b) a PCMB-communication unit configured to communicate with any device sharing same the communication protocols;
c) an indicator; and
d) an RFID reader,
wherein said processing unit is configured to analyze the detected hand motions of the health care worker to thereby determine if said hand motion were performed in compliance with a preset required hand motion sequence and rate.
wherein said PCMB-communication unit is configured to communicate with said at least one smart-wrist-wearable device and said data repository unit;
wherein said data repository unit includes a data base of known hand motions, wherein hand motion analysis includes matching said detect hand motions with said known hand motions; and
wherein said personal-communication-monitoring-badge device is configured to perform a hand motion analysis to thereby analyze the detected hand motions of the health care worker, and to thereby determine if said hand motion were performed in compliance with a preset required hand motion sequence and rate.

10. The infection-control-practices-monitoring system of claim 9, wherein said at least one personal-communication-monitoring-badge device further includes an imaging device.

11. The infection-control-practices-monitoring system of claim 9, wherein said at least one personal-communication-monitoring-badge device further includes a UV emitting light.

12. The infection-control-practices-monitoring system of claim 9, wherein said at least one personal-communication-monitoring-badge device further includes a UV sensor.

13. The infection-control-practices-monitoring system of claim 10, wherein said imaging device is an IR imaging device.

14. The infection-control-practices-monitoring system of claim 13, wherein said IR imaging device includes a wide-angle lens.

15. The infection-control-practices-monitoring system of claim 1 further comprising at least one Hand Hygiene bottle containing Hand Hygiene solution, wherein said Hand Hygiene bottle comprises an RFID tag, and wherein said RFID reader of said smart-wrist-wearable device is configured to read said RFID tag of said Hand Hygiene bottle.

16. The infection-control-practices-monitoring system of claim 15 further comprising at least one Hand Hygiene bottle containing Hand Hygiene solution, wherein said Hand Hygiene bottle comprises an RFID tag, and wherein said RFID reader of said smart-wrist-wearable device is configured to read said RFID tag of said Hand Hygiene bottle.

17. The infection-control-practices-monitoring system of claim 9 further comprising at least one Hand Hygiene bottle containing Hand Hygiene solution, wherein said Hand Hygiene bottle comprises an RFID tag, and wherein said RFID reader of said personal-communication-monitoring-badge device is configured to read said RFID tag of said Hand Hygiene bottle.

18. The infection-control-practices-monitoring system of claim 9, wherein said at least one smart-wrist-wearable device includes a living body proximity sensor, an accelerometer, a gyroscope and a communication unit for communicating with said personal-communication-monitoring-badge device.

19. The infection-control-practices-monitoring system of claim 1, wherein said preset required hand motion sequence and rate are selected from the group of procedures put forward by the World Health Organization” (WHO), including “Your 5 Moments Hand Hygiene”, “How to Handwash”, “How to Handrub”, “technique for donning and removing non-sterile examination gloves” and “Putting on and removing PPE”.

20. A smart-wrist-wearable device for detecting and monitoring hand motions of a health care worker (HCW), the hand motions are related to infection control practices, the smart-wrist-wearable device comprising:
a) an accelerometer;
b) a gyroscope;
c) a living body proximity sensor; and
d) a bracelet-communication unit,
wherein the at least one smart-wrist-wearable device adapted to be worn on a human wrist of a first hand;
wherein said bracelet-communication unit is configured to communicate with a data repository unit;
wherein said living body proximity sensor is configured to detect a live tissue at a short range of up to 50 centimeters;
wherein at least one of said accelerometer, said gyroscope and said living body proximity sensor detects hand motions of the health care worker; and
wherein said smart-wrist-wearable device is configured to perform a hand motion analysis to thereby analyze the detected hand motions of the health care worker, and to thereby determine if said hand motion were performed in compliance with a preset required hand motion sequence and rate.

21. The smart-wrist-wearable device of claim 20, wherein said data repository unit includes a data base of known hand motions, and wherein said hand motion analysis includes matching said detect hand motions with said known hand motions.

22. The smart-wrist-wearable device of claim 20, wherein said at least one smart-wrist-wearable device further includes a skin and muscle motion detector for detecting under-skin muscle motion.

23. The smart-wrist-wearable device of claim 20, wherein said at least one smart-wrist-wearable device further includes a RFID reader.

24. The smart-wrist-wearable device of claim 20, wherein said at least one smart-wrist-wearable device further includes an indicator.

25. The smart-wrist-wearable device of claim 20 further includes a UV sensor.

26. (canceled)

27. The smart-wrist-wearable device of claim 20, wherein said living body proximity sensor is selected from group including a thermal sensor, an RLC sensor, an ultrasound sensor and a combination thereof

28. The smart-wrist-wearable device of claim 20, wherein said living body proximity sensor is capable of sensing the second hand of the HCW.

29. The smart-wrist-wearable device claim 20, wherein said preset required hand motion sequence and rate are selected from the group of procedures put forward by the World Health Organization” (WHO), including “Your 5 Moments Hand Hygiene”, “How to Handwash”, “How to Handrub”, “technique for donning and removing non-sterile examination gloves” and “Putting on and removing PPE”.

30. A personal-communication-monitoring-badge device for monitoring hand motions of a health care worker (HCW), the hand motions are related to infection control practices, the personal-communication-monitoring-badge device comprising:
a) a processing unit;
b) a PCMB-communication unit;
c) an indicator; and
d) an RFID reader,
wherein the hand motions are detected by the smart-wrist-wearable device as in claim at wherein said processing unit is configured to analyze the detected hand motions of the health care worker to thereby determine if said hand motion were performed in compliance with a preset required hand motion sequence and rate.
wherein said PCMB-communication unit is configured to communicate with at least one smart-wrist-wearable device and a data repository unit;
wherein said at least one smart-wrist-wearable device adapted to be worn on a human wrist of a first hand and is configured to communicate with said PCMB-communication unit;
wherein said bracelet-communication unit includes a multiplicity of sensors configured at least detect hand motions of the HCW and proximity of the two wrists of the HCW;
wherein said data repository unit includes a data base of known hand motions, wherein hand motion analysis includes matching said detect hand motions with said known hand motions; and
wherein said personal-communication-monitoring-badge device is configured to perform a hand motion analysis to thereby analyze the detected hand motions of the health care worker, and to thereby determine if said hand motion were performed in compliance with a preset required hand motion sequence and rate.

31. The personal-communication-monitoring-badge device of claim 30, wherein said at least one personal-communication-monitoring-badge device further includes an imaging device.

32. The personal-communication-monitoring-badge device of claim 30, wherein said at least one personal-communication-monitoring-badge device further includes a UV emitting light.

33. The personal-communication-monitoring-badge device of claim 30, wherein said at least one personal-communication-monitoring-badge device further includes a UV sensor.

34. The personal-communication-monitoring-badge device of claim 31, wherein said imaging device is an IR imaging device.

35. The personal-communication-monitoring-badge device of claim 34, wherein said IR imaging device includes a wide angle lens.

36. An infection-control-practices-monitoring method for monitoring hand motions of a HCW while performing a Hand Hygiene (HH) according to the WHO Handwash/Handrub procedures, the method comprising the steps of:
a) providing an infection-control-practices-monitoring system as in claim 9.
b) identifying the patient's room;
c) determining that the HCW has used an HH solution;
d) monitoring hand motions of the HCW performing the Hand Hygiene, in each step of the HH procedure;
e) determining compliance with the WHO Handwash/Handrub procedure;
f) providing an indication to the HCW and the patient to indicate success or failure of the HH procedure; and
g) recording to said performed HH procedure at said data repository unit,
wherein said determining compliance motions includes determining proximity of the two wrists of the HCW, while performing the Hand Hygiene procedure.

37. The infection-control-practices-monitoring method of claim 36, wherein said monitoring hand motions includes all motions required by the WHO Handwash/Handrub procedures, while performing the Hand Hygiene procedure.

38. The infection-control-practices-monitoring system of claim 9 further comprising a training sub-system comprising:
a) a computerized device configured to track the training progress and performance level of Hand Hygiene procedures of a multiplicity of health care workers;
b) at least one indicator;
c) a Hand Hygiene bottle having an RFID tag;
d) a Hand-Hygiene hand motions database associated with said data repository unit;
e) a non-Hand-Hygiene hand motions database associated with said data repository unit;
and
f) a health-care-workers database associated with said data repository unit,
wherein said computerized device comprises a machine learning module that is configured to learn a particular HCW's variations of hand motions;
wherein said machine learning module is configured to track and evaluate the progress of a HCW; and
wherein said computerized device is configured to communicate with said PCMB-communication unit and with said data repository unit and said at least one smart-wrist-wearable device.

39. The infection-control-practices-monitoring system of claim 38, wherein said machine learning module forms a uniquely identifiable hand motion model associated with a particular HCW, based on that HCW specific hand hygiene motions or combination of some of that HCW specific hand hygiene motions.

40. The infection-control-practices-monitoring system of claim 38 further comprises a second smart-wrist-wearable device adapted to be worn on a human wrist of the second hand.

41. The infection-control-practices-monitoring system of claim 38 further comprises an alcoholic hand rub bottle (AHRB) usage sensor for determining that the nozzle of the AHRB is being used.

42. (canceled)

43. (canceled)

44. (canceled)

45. The infection-control-practices-monitoring method of claim 36 further including training steps for monitoring hand motions of a HCW while putting on and removing personal protective equipment (PPE) according to the WHO PPE procedures.

46. The infection-control-practices-monitoring method of claim 36 further including training steps for monitoring hand motions of a HCW while donning and removing non-sterile examination gloves according to the WHO glove procedures.
